(19) United States
(12) Patent Application Publication
(10) Pub. No.: US 2013/0059021 A1
(43) Pub. Date: Mar. 7, 2013

(54) PERFLUORO(N-BUTYLCYCLOHEXANE) COMPOSITIONS AND USES THEREOF

(71) Applicants: Aharon Grossman, Morrisville, NC (US); Chris Stern, Raleigh, NC (US); Richard Kiral, Costa Mesa, CA (US); Gary Clauson, Costa Mesa, CA (US); Gerald Klein, Morrisville, NC (US)

(72) Inventors: Aharon Grossman, Morrisville, NC (US); Chris Stern, Raleigh, NC (US); Richard Kiral, Costa Mesa, CA (US); Gary Clauson, Costa Mesa, CA (US); Gerald Klein, Morrisville, NC (US)

(73) Assignee: Oxygen Biotherapeutics, Inc.

(21) Appl. No.: 13/605,586

(22) Filed: Sep. 6, 2012

Related U.S. Application Data
(63) Continuation of application No. 13/398,576, filed on Feb. 16, 2012, now abandoned.
(60) Provisional application No. 61/443,664, filed on Feb. 16, 2011.

(51) Int. Cl. A61K 31/025; A61K 36/63; A61P 17/00; A61Q 19/08; A61K 8/30; A61P 17/04; A61P 17/06; A61P 17/10; A61K 8/97; A61K 8/60; A61K 31/695; A61P 17/02

(52) U.S. Cl. 424/769; 514/747; 514/26; 514/63

(57) ABSTRACT

A perfluoro(n-butylcyclohexane) composition is disclosed with numerous uses including topical and cosmetic applications, e.g., for application to the periocular skin or for the topical treatment of pruritus.

This application claims benefit of U.S. Provisional Application No. 61/443,664, filed Feb. 16, 2011, the entire content of which is hereby incorporated by reference herein.

Throughout this application various publications, published patent applications, and patents are referenced. The disclosures of these documents in their entireties are hereby incorporated by reference into this application in order to more fully describe the state of the art to which this invention pertains.

### BACKGROUND OF THE INVENTION

Perfluorocarbons (PFCs) possess the ability to dissolve large quantities of many gases at concentrations much larger than water, saline and plasma. In addition, PFCs can transport these gases to diffuse across distances. Thus, PFCs can be a convenient and inexpensive means to deliver high levels of oxygen or other therapeutic gases to tissues and organ systems.

PFCs that are commonly used in medical research are non-toxic, biologically inert, biostatic liquids at room temperature with densities of about 1.5-2.0 g/mL and high solubilities for oxygen and carbon dioxide. Such PFCs have been found to be efficient carriers of gases, both as emulsions for intravenous use and as neat liquids for liquid ventilation applications.

### Periocular Skin

The skin around the eyes, or periocular skin, is among the most delicate areas of the body, and is liable to show the signs of aging, including wrinkles, fine lines and dark under-eye circles, before the rest of the face.

Periocular skin is distinct from other parts of the skin. The differences are notably that skin in this area contains less lipid in the corneum stratum, the outermost layer of the epidermis, that the corneum stratum has fewer layers, that it has higher epidermal kinetics and that it is located close to a warm and moist environment. In addition to being thinner than skin in other area of the body, periocular skin also contains fewer oil glands. These characteristics make periocular skin especially sensitive and vulnerable to damage from various sources, including environmental damages and aging.

The skin around the eyes is also a difficult area of skin to care for.

### Pruritus

Pruritus (itching) is a sensation that a patient instinctively attempts to relieve by scratching or rubbing. It is a symptom and not a disease, and may accompany a primary skin disease or a systemic disease. (The Merck Manual, 1999.)

Skin diseases causing severe pruritus vary, and include scabies, pediculosis, insect bites, urticaria, atopic dermatitis, contact dermatitis, lichen planus, miliaria, and dermatitis herpetiformis. Dry skin often causes severe generalized pruritus. (The Merck Manual, 1999.)

Systemic conditions that cause generalized pruritus, usually without skin lesions, include obstructive biliary disease, uremia (frequently associated with hyperparathyroidism), lymphomas, leukemias, and polycythemia rubra vera. Pruritus may also occur during the later months of pregnancy. Many drugs (especially barbiturates and salicylates) can cause pruritus. Less well-defined associations with generalized pruritus include hyperthyroidism, diabetes mellitus, and internal cancers of many types. Pruritus is uncommonly purely psychogenic. (The Merck Manual, 1999.)

Persistent scratching may produce redness, linear urticarial papules, excoriation of preexisting papules, fissures, and elongated crusts along scratch lines, which may obscure the underlying disease. Lichenification and pigmentation may also result from prolonged scratching and rubbing. Occasionally, patients who complain of severe generalized pruritus have few signs of scratching or rubbing the skin. (The Merck Manual, 1999.)

Conventionally caine-based anesthetics are avoided, but lotions or creams containing 0.125% to 0.25% menthol can be useful. Ultraviolet B to the skin and oral cholestyramine can be helpful in uremia and cholestasis and at times in undiagnosed cases. Topical corticosteroids seldom alleviate generalized pruritus (without dermatitis) but may uncommonly be useful if used with lubricants in elderly patients with dry skin. (The Merck Manual, 1999.)

If a drug has been ruled out as the cause of pruritus, hydroxyzine (10 to 50 mg po q 4 h prn) can be prescribed or, for more severe cases, minimal and gradually increasing doses of trimeprazine or the antidepressant doxepin. If antihistamines are helpful, their sedative effect may be the reason. Antihistamines are more likely to cause intolerable side effects in the elderly. More recently several newer low-sedating antihistamines have become available, including astemizole, loratadine, and cetirizine. These drugs have been used with limited success in the treatment of pruritus. (The Merck Manual, 1999.)

Given the wide range of conditions which can cause pruritus, and the lack of a single effective therapy, additional therapies for pruritus are needed.

### Dermatitis (Eczema)

Dermatitis is superficial skin inflammation, characterized histologically by epidermal edema and clinically by vesicles (when acute), poorly marginated redness, edema, oozing, crusting, scaling, usually pruritus, and lichenification caused by scratching or rubbing. (The Merck Manual, 1999.) As noted above, dermatitis (eczema) usually causes pruritus.

Authorities generally disagree about how to use the synonymous terms eczema and dermatitis. Often eczema refers to vesicular dermatitis, but some authorities restrict eczema to mean chronic dermatitis. Some also refer to dermatitis as spongiotic dermatitis because spongiosis (intraepidermal edema) is a histologic feature. (The Merck Manual, 1999.)

Dermatitis includes contact dermatitis and atopic dermatitis. Contact dermatitis is the acute or chronic inflammation, often asymmetric or oddly shaped, produced by substances contacting the skin and causing toxic (irritant) or allergic reactions. Atopic dermatitis is the chronic, pruritic, superficial inflammation of the skin, frequently associated with a personal or family history of allergic disorders (e.g., hay fever, asthma). (The Merck Manual, 1999.)

### Psoriasis

Psoriasis is a common chronic, recurrent disease characterized by dry, well-circumscribed, silvery, scaling papules and plaques of various sizes. Psoriasis varies in severity from one to two lesions to widespread dermatosis, sometimes associated with disabling arthritis or exfoliation. The cause is unknown, but the thick scaling has traditionally been attributed to increased epidermal cell proliferation and concomitant dermal inflammation. The response of psoriasis to the immunosuppressive drug cyclosporine suggests that the primary pathogenetic factor may be immunologic. (The Merck Manual, 1999.) Psoriasis causes pruritus.

Conventionally, lubricants, keratolytics, topical corticosteroids, topical vitamin D derivatives and anthralin are tried first in patients with a limited number of lesions. Exposure to sunlight is also beneficial though occasionally sunburn may induce exacerbations. Systemic antimetabolites (e.g., methotrexate) are used only in patients with severe skin Or joint involvement. Immunosuppressive drugs (e.g., cyclosporine, tacrolimus, mycophenolate mofetil) have been used in severe and recalcitrant cases, but these drugs are not currently approved in the U.S. for treatment of psoriasis. Systemic corticosteroids should not be used because side effects, including severe exacerbations or pustular lesions, may occur during treatment (even with increasing doses) or after treatment. (The Merck Manual, 1999.)

### Acne

Acne is a dermatological condition that is thought to be caused by genetic factors, increased sebum production, abnormal keratinization of the hair follicle, host immune response, and due to the harmful effects of increased proliferation of the anaerobic bacteria Propionibacterium acnes. This type of bacteria is responsible for much of the inflammatory reaction that occurs in acne, thought to be due to its release of toxins. Inflammation occurs when P. acnes, growing in plugged follicles, releases chemoattractants eliciting the inflammatory response creating the classical comedones of acne. Therefore, the clinical manifestations appear to be the result of bacterial-induced inflammation of a plugged sebaceous gland. Inflammation is further enhanced by follicular rupture and subsequent leakage of lipids, bacteria, and fatty acids into the dermis. Systemic and topical antibiotics are used for both treatment and prophylaxis of acne. Treatments that reduce P. acnes numbers lead to clinical improvement of acne (Thiboutot, 1997) and, finally, to the emergence of antibiotic-resistant P. acnes strains are linked to the failure of antibiotic treatment (Eady et al, 1989).

Current treatment of acne consists of selection of a topical therapy which is based on the severity and type of acne. Topical retinoids, benzoyl peroxide, and azelaic acid are effective treatments for mild acne. Topical tretinoin (Retin-A) which is a derivative of vitamin A, and a comedolytic agent that normalizes desquamation of the epithelial lining, thereby preventing obstruction of the pilosebaceous outlet. This agent also appears to have direct anti-inflammatory effects. Topical antibiotics and medications with bacteriostatic and anti-inflammatory properties are effective for treating mild to moderate inflammatory acne. Systemic antibiotics are used for the moderate to severe patient. Isotretinoins is used to treat severe, often nodulocystic and inflammatory acne. Isotretinoin (Accutane) acts against the four pathogenic factors that contribute to acne. It is the only medication with the potential to suppress acne over the long term. To be able to prescribe this medication, the physician must be a registered member of the manufacturer's System to Manage Accutane-Related Teratogenicity (SMART) program. The SMART program was developed in conjunction with the U.S. Food and Drug Administration (FDA) to minimize unwanted pregnancies and educate patients about the possible severe adverse effects and teratogenicity of isotretinoin, which is a pregnancy category X drug.

### SUMMARY OF THE INVENTION

The subject application provides for a perfluorocarbon composition comprising 1-90 wt % perfluoro(n-butylcyclohexane) relative to the total weight of the composition.

The subject application also provides for a perfluorocarbon composition comprising 1) perfluoro(n-butylcyclohexane), 2) dipotassium glycyrrhizate, and 3) a mixture comprising phenoxyethanol, caprylyl glycol and chlorphenesin.

The subject application also provides for a perfluorocarbon composition comprising perfluoro(n-butylcyclohexane), cetyl phosphate and cyclopentasiloxane.

The subject application also provides for a perfluorocarbon composition comprising 1) perfluoro(n-butylcyclohexane), 2) ascorbyl glucoside, 3) a first mixture comprising butylene glycol, water, niacinamide, Fraxinus excelsior bark extract, silanetriol, and potassium citrate, 4) a second mixture comprising water, glycerin, steareth-20, N-hydroxysuccinimide, chrysin, palmitoyl oligopeptide and palmitoyl tetrapeptide-7 and 5) a third mixture comprising glycerin, water, butylene glycol, carbomer, polysorbate 20, palmitoyl oligopeptide, and palmitoyl tetrapeptide-7.

The subject application also provides for a method of delivering oxygen to the skin of a subject comprising topically administering to the skin the claimed perfluorocarbon compositions effective to deliver oxygen to the skin.

The subject application also provides for a method of increasing the firmness of the skin or reducing the appearance of fine lines, wrinkles or scars in a subject comprising topically administering to the skin of the subject the claimed perfluorocarbon compositions effective to increase the firmness of the subject's skin or reduce the appearance of fine lines, wrinkles or scars on the subject's skin.

The subject application also provides for a method of improving the appearance of the skin of a subject comprising topically administering the skin the claimed perfluorocarbon compositions effective to improve the appearance of the skin.

The subject application also provides for a method of treating a wound, a burn injury, pruritus, psoriasis, acne or rosacea in a subject suffering therefrom comprising topically administering to the skin of a subject the claimed perfluorocarbon compositions effective to treat the subject's wound, burn injury, pruritus, psoriasis, acne or rosacea.

The subject application further provides the disclosed perfluorocarbon composition for use in delivering oxygen to the skin of a subject, for use in increasing the firmness of the skin or reducing the appearance of fine lines, wrinkles or scars in a subject, for use in improving the appearance of the skin of a subject, and for use in treating a wound, a burn injury, pruritus, psoriasis, acne or rosacea in a subject suffering therefrom.

### DETAILED DESCRIPTION OF THE INVENTION

### Embodiments of the Invention

The subject application provides for a perfluorocarbon composition comprising 1-90 wt % perfluoro(n-butylcyclohexane) relative to the total weight of the composition.

In one embodiment, the perfluorocarbon composition further comprises 8-70 wt % water relative to the total weight of the composition. In another embodiment, the composition further comprises 1-5 wt % surfactants. In another embodiment, the surfactants include polyoxyethylene-polyoxypropylene block copolymers. In another embodiment, the polyoxyethylene-polyoxypropylene block copolymers include Poloxamer 105 and/or Poloxamer 188.

In one embodiment, the composition further comprises 0.01-10 wt % Vitamin E. In another embodiment, the composition comprises 0.03 wt % Vitamin E.

In one embodiment, the composition further comprises 0.02-3.20 wt % preservatives. In another embodiment, the preservatives include poly(diallyldimethylammonium chloride), poly(acrylamide-co-diallyldimethylammonium chloride) and/or ethylene diamine tetraacetic acid.

In one embodiment, the composition further comprises 0.10⁻² wt % copper. In another embodiment, the copper is copper (II) oxide.

In one embodiment, the composition comprises 90 wt % perfluoro(n-butylcyclohexane), 8 wt % water, and 2 wt % surfactants. In another embodiment, the composition comprises 30-50 wt % perfluoro(n-butylcyclohexane), 48-70 wt % water, and 2 wt % surfactants. In another embodiment, the composition comprises 86.86 wt % perfluoro(n-butylcyclohexane), 10.42 wt % water, 2.69 wt % surfactants and 0.03 wt % Vitamin E. In yet another embodiment, the composition comprises 86.86 wt % perfluoro(n-butylcyclohexane), 10.42 wt % water, 2.43 wt % Poloxamer 105, 0.26 wt % Poloxamer 188 and 0.03 wt % Vitamin E.

In one embodiment, the preservatives include 0-0.40 wt % poly(diallyldimethylammonium chloride), 0.01-0.80 wt % poly(acrylamide-co-diallyldimethylammonium chloride) and 0.01-2.00 wt % ethylene diamine tetraacetic acid. In another embodiment, the composition comprises 84-88 wt % perfluoro(n-butylcyclohexane), 9-11 wt % water, 2-3 wt % Poloxamer 105, 0.01-1 wt % Poloxamer 188, 0-0.40 wt % poly(diallyldimethylammonium chloride), 0.01-0.80 wt % poly(acrylamide-co-diallyldimethylammonium chloride) and 0.01-2.00 wt % ethylene diamine tetraacetic acid.

In one embodiment, the composition comprises 85.98 wt % perfluoro(n-butylcyclohexane), 10.28 wt % water, 2.45 wt % Poloxamer 105, 0.31 wt % Poloxamer 188, 0.74 wt % poly(acrylamide-co-diallyldimethylammonium chloride) and 0.25 wt % ethylene diamine tetraacetic acid.

In one embodiment, the composition comprises 86.73 wt % perfluoro(n-butylcyclohexane), 10.37 wt % water, 2.47 wt % Poloxamer 105, 0.31 wt % Poloxamer 188, 0.10 wt % poly(acrylamide-co-diallyldimethylammonium chloride) and 0.03 wt % ethylene diamine tetraacetic acid.

In one embodiment, the composition comprises 85.98 wt % perfluoro(n-butylcyclohexane), 10.28 wt % water, 2.45 wt % Poloxamer 105, 0.31 wt % Poloxamer 188, 0.25 wt % poly(diallyldimethylammonium chloride), 0.50 wt % poly(acrylamide-co-diallyldimethylammonium chloride) and 0.25 wt % ethylene diamine tetraacetic acid.

In one embodiment, the composition comprises 86.73 wt % perfluoro(n-butylcyclohexane), 10.37 wt % water, 2.47 wt % Poloxamer 105, 0.31 wt % Poloxamer 188, 0.03 wt % poly(diallyldimethylammonium chloride), 0.07 wt % poly(acrylamide-co-diallyldimethylammonium chloride) and 0.03 wt % ethylene diamine tetraacetic acid.

In one embodiment, the perfluorocarbon composition is characterized by that it continuously delivers oxygen to a tissue at a rate of 0.2 cc/hour −20.0 cc/hour for up to 24 hours. In another embodiment, the perfluorocarbon composition continuously delivers oxygen to the tissue at a rate of 2.0 cc/hour for 24 hours. In yet another embodiment, the perfluorocarbon composition further comprises urea hydrogen peroxide.

The subject application also provides for a perfluorocarbon composition comprising 1) perfluoro(n-butylcyclohexane), 2) dipotassium glycyrrhizate and 3) a mixture comprising phenoxyethanol, caprylyl glycol and chlorphenesin. In one embodiment, the perfluorocarbon composition further comprises a second mixture comprising sorbitan olivate and cetearyl olivate.

In one embodiment, the perfluorocarbon composition comprises 1-10 wt % perfluoro(n-butylcyclohexane), 0.05-1 wt % dipotassium glycyrrhizate, 0.1-5 wt % of the mixture comprising phenoxyethanol, caprylyl glycol and chlorphenesin, 0.01-5 wt % of the second mixture comprising sorbitan olivate and cetearyl olivate and 15-50 wt % water.

In another embodiment, the perfluorocarbon composition comprises perfluoro(n-butylcyclohexane), water, Alaria esculenta extract, aminomethyl propanol, ascorbyl palmitate, butylene glycol, Butyrospermum parkii (shea butter), caprylic/capric triglyceride, caprylyl glycol, carbomer, cetyl alcohol, cetearyl olivate, chlorphenesin, dimethicone, dimethicone crosspolymer, dipotassium glycyrrhizate, disodium EDTA, glycerin, glyceryl stearate, Olea europaea (olive) fruit oil, palmitoyl oligopeptide, palmitoyl tetrapeptide-7, PEG-100 stearate, Persea gratissima (avocado) Oil, phenoxyethanol, phospholipids, phytosterols, polysorbate 20, propanediol, retinyl palmitate, Simmondsia chinensis (jojoba) seed oil, sorbitan olivate and tocopheryl acetate.

The subject application also provides for a perfluorocarbon composition comprising perfluoro(n-butylcyclohexane), cetyl phosphate and cyclopentasiloxane.

In an embodiment the perfluorocarbon composition further comprises isopropyl isostearate. In another embodiment, the perfluorocarbon composition comprises 35-60 wt % perfluoro(n-butylcyclohexane), 0.5-5 wt % cetyl phosphate, 1-5 wt % cyclopentasiloxane, 1-5 wt % isopropyl isostearate and 35-60 wt % water. In a further embodiment, the perfluorocarbon composition comprises perfluoro(n-butylcyclohexane), water, acrylates/C₁₀-C₃₀ alkyl acrylate crosspolymer, aminomethyl propanol, Bambusa vulgaris leaf/stern extract, caprylyl glycol, cetyl phosphate, cyclopentasiloxane, disodium EDTA, ethylhexylglycerin, glucosemine HCl, hexylene glycol, isopropyl isostearate, pentylene glycol, phenoxyethanol, Pisum sativum (Pea) extract, and sodium PCA.

In yet another embodiment, the perfluorocarbon composition comprises perfluoro(n-butylcyclohexane), water, ascorbic glucoside, butylene glycol, Butyrospermum parkii (shea butter), caprylic/capric triglyceride, caprylyl glycol, cellulose gum, cetearyl alcohol, cetearyl glucoside, cetearyl olivate, chlorphenesin, Crithmum maritimum extract, dipotassium glycyrrhizate, disodium EDTA, glycerin, glycine soja (soybean) oil, heptyl undecylenate, hydrogenated lecithin, hydrogenated olive oil, hydrogenated vegetable oil, Limnanthes alba (meadowfoam) seed oil, microcrystalline cellulose, Olea europaea (olive) fruit oil, Olea europaea (olive) oil unsaponifiables, oligopeptide-68, phenoxyethanol, propanediol, sodium citrate, sodium hydroxide, sodium oleate, sodium phytate, sorbitan olivate, tetrahexyldecyl ascorbate and xanthan gum.

The subject application also provides for a perfluorocarbon composition comprising 1) perfluoro(n-butylcyclohexane) 2) ascorbyl glucoside, 3) a first mixture comprising butylene glycol, water, niacinamide, Fraxinus excelsior bark extract, silanetriol, and potassium citrate, 4) a second mixture comprising water, glycerin, steareth-20, N-hydroxysuccinimide, chrysin, palmitoyl oligopeptide and palmitoyl tetrapeptide-7 and 5) a third mixture comprising glycerin, water, butylene glycol, carbomer, polysorbate 20, palmitoyl oligopeptide, and palmitoyl tetrapeptide-7.

In one embodiment, the composition comprises 3-90 wt % perfluoro(n-butylcyclohexane) relative to the total weight of the composition. In another embodiment, the composition comprises 5-90 wt % perfluoro(n-butylcyclohexane) relative to the total weight of the composition. In another embodiment, the composition comprises 15-90 wt % perfluoro(n-butylcyclohexane) relative to the total weight of the composition. In another embodiment, the composition comprises 1-55 wt % perfluoro(n-butylcyclohexane) relative to the total weight of the composition. In another embodiment, the composition comprises 3-55 wt % perfluoro(n-butylcyclohexane) relative to the total weight of the composition. In another embodiment, the composition comprises 3-10 wt % perfluoro(n-butylcyclohexane) relative to the total weight of the composition. In another embodiment, the composition comprises the perfluorocarbon is 17-25 wt % perfluoro(n-butylcyclohexane) relative to the total weight of the composition. In another embodiment, the composition comprises 45-55 wt % perfluoro(n-butylcyclohexane) relative to the total weight of the composition. In another embodiment, the composition comprises 25 wt % perfluoro(n-butylcyclohexane) relative to the total weight of the composition. In another embodiment, the composition comprises 50 wt % perfluoro(n-butylcyclohexane) relative to the total weight of the composition.

In one embodiment, the ascorbyl glucoside is 1-10 wt % relative to the total weight of the composition. In another embodiment, the first mixture is 1-10 wt % relative to the total weight of the composition. In another embodiment, the second mixture is 1-10 wt % relative to the total weight of the composition. In another embodiment, the third mixture is 1-10 wt % relative to the total weight of the composition.

In one embodiment, the perfluorocarbon composition comprises 1) 15-50 wt % perfluoro(n-butylcyclohexane), 2) 0.1-5 wt % ascorbyl glucoside, 3) 1-10 wt % of the first mixture comprising butylene glycol, water, niacinamide, Fraxinus excelsior bark extract, silanetriol, and potassium citrate, 4) 0.1-5 wt % of the second mixture comprising water, glycerin, steareth-20, N-hydroxysuccinimide, chrysin, palmitoyl oligopeptide and palmitoyl tetrapeptide-7, 5) 0.1-5 wt % of the third mixture comprising glycerin, water, butylene glycol, carbomer, polysorbate 20, palmitoyl oligopeptide, and palmitoyl tetrapeptide-7, and 6) 15-50 wt % water.

In another embodiment, the perfluorocarbon composition comprises perfluoro(n-butylcyclohexane), water, acrylates/cimethicone copolymer, ascorbyl glucoside, Avena sativa (oat) kernel extract, butylene glycol, Butyrospermum parkii (shea butter), caprylic/capric triglyceride, caprylyl glycol, carbomer, cetyl phosphate, chlorphenesin, chrysin, cyclopentasiloxane, dimethicone, dipotassium glycyrrhizate, disodium EDTA, fragrance (parfum) #6110985, Fraxinus excelsior bark extract, glycerin, glyceryl stearate, Green 5 [C.I. 61570], Helianthus annuus (sunflower) seed oil unsaponifiables, Limnanthes alba (meadowfoam) seed oil, N-hydroxysuccinimide, niacinamide, Olea europaea (olive) fruit oil, palmitoyl oligopeptide, palmitoyl tetrapeptide-7, PEG-100 stearate, Persea gratissima (avocado) oil, phenoxyethanol, phytosterols, polysorbate 20, polyurethane-40, potassium citrate, propanediol, silanetriol, silica, sodium hydroxide, steareth-20 and stearic acid.

In one embodiment, the perfluorocarbon composition is characterized by it having a viscosity of 5,000-80,000 cps at 25° C. In a further embodiment, the perfluorocarbon composition is characterized by it having a viscosity of 5,000-30,000 cps at 25° C. In another embodiment, the composition is characterized by it having a viscosity of 10,000-20,000 cps at 25° C. In another embodiment, the composition is characterized by it having a viscosity of 10,000-30,000 cps at 25° C. In another embodiment, the composition is characterized by it having a viscosity of 13,000-18,000 cps at 25° C. In another embodiment, the composition is characterized by it having a viscosity of 18,000-30,000 cps at 25° C. In another embodiment, the composition is characterized by it having a viscosity of 15,000-25,000 cps at 25° C. In another embodiment, the composition is characterized by it having a viscosity of 20,000-30,000 cps at 25° C. In another embodiment, the composition is characterized by it having a viscosity of 35,000-75,000 cps at 25° C.

In one embodiment, the composition is characterized by it having a specific gravity of 0.9-1.82. In another embodiment, the composition is characterized by it having a specific gravity of 1.01-1.82. In another embodiment, the composition is characterized by it having a specific gravity of 1.14-1.18. In another embodiment, the composition is characterized by it having a specific gravity of 1.02-1.22. In another embodiment, the composition is characterized by it having a specific gravity of 0.97-1.01. In another embodiment, the composition is characterized by it having a specific gravity of 1.01-1.04. In another embodiment, the composition is characterized by it having a specific gravity of 1.26-1.34.

In one embodiment, the perfluorocarbon composition further comprises a pharmaceutically acceptable carriers or a cosmetic carrier. In another embodiment, the perfluorocarbon composition is in the form of a liquid, cream, lotion or gel. In another embodiment, the composition further comprises component or components that absorbs or reflects some of the sun's ultraviolet (UV) radiation on the skin exposed to sunlight and thus helps protect against sunburn. In one embodiment, the component is capable of boosting sun protection factor (SPF).

The subject application also provides for a method of continuously delivering oxygen to a tissue at a rate of 0.2 cc/hour −20.0 cc/hour for up to 24 hours by contacting the tissue with a perfluorocarbon composition described herein.

The subject application also provides for a method of delivering oxygen to the skin of a subject comprising topically administering to the skin the claimed perfluorocarbon compositions effective to deliver oxygen to the skin.

The subject application also provides for a method of increasing the firmness of the skin or reducing the appearance of fine lines, wrinkles or scars in a subject comprising topically administering to the skin of the subject the claimed perfluorocarbon compositions effective to increase the firmness of the subject's skin or reduce the appearance of fine lines, wrinkles or scars on the subject's skin.

The subject application also provides for a method of improving the appearance of the skin of a subject comprising topically administering the skin the claimed perfluorocarbon compositions effective to improve the appearance of the skin. In one embodiment, the administration of the claimed perfluorocarbon compositions brightens the subject's complexion. In another embodiment, the administration of the claimed perfluorocarbon composition reduces the appearance of dullness of the subject's skin.

In one embodiment, the skin is periocular skin and the administration is to a facial area consisting of the periocular skin of the subject

In another embodiment, the improvement in appearance is the reduction of the severity of fine lines, wrinkles, skin elastosis, puffiness, dark circles, under-eye circles, bags and/or dark blemishes.

In one embodiment, the composition is administered periodically. In another embodiment, the composition is administered twice daily. In another embodiment, the administration is for a period of greater than 3 weeks. In yet another embodiment, the administration is for a period of 8 weeks or more.

In one embodiment, the subject's Fitzpatrick Wrinkle Assessment Scale score is decreased. In another embodiment, the subject's Fitzpatrick Wrinkle Assessment Scale score is decreased by at least 1 point. In another embodiment, the subject's Fitzpatrick Wrinkle Assessment Scale score is decreased by at least 2 points. In yet another embodiment, the subject's Global Aesthetic Improvement Scale score is improved.

The subject application also provides for a method of treating a wound, a burn injury, pruritus, psoriasis, acne or rosacea in a subject suffering therefrom comprising topically administering to the skin of a subject the claimed perfluorocarbon compositions effective to treat the subject's wound, burn injury, pruritus, psoriasis, acne or rosacea.

In one embodiment, the composition is administered periodically. In another embodiment, the composition is administered twice daily. In another embodiment, the administration is for a period of greater than 3 weeks. In yet another embodiment, the administration is for a period of 8 weeks or more.

In one embodiment, the pruritus is induced by histamine. In another embodiment, the pruritus is a symptom of an inflammatory skin condition, xerosis, a dermatological allergic response, allergic dermatitis, atopic dermatitis or contact dermatitis. In another embodiment, the inflammatory skin condition is psoriasis.

In one embodiment, the perfluorocarbon composition is administered topically to the portion of the subject's skin afflicted with the pruritus or psoriasis.

In one embodiment, the subject is afflicted with edema, erythema or erythematous lesion. In another embodiment, the administration of the perfluorocarbon reduces the edema. In another embodiment, the administration of the perfluorocarbon reduces the erythema. In another embodiment, the administration of the perfluorocarbon reduces the erythematous lesion. In yet another embodiment, the administration of the perfluorocarbon composition reduces subject-perceived itching.

In one embodiment, the administration of the perfluorocarbon composition reduces subject-perceived itching. In another embodiment, the administration of the perfluorocarbon composition relieves the subject's pruritus for 1-6 hours. In another embodiment, the administration of the perfluorocarbon composition relieves the subject's pruritus for 2 hours or more. In another embodiment, the administration of the perfluorocarbon composition relieves the subject's pruritus for 3 hours or more. In yet another embodiment, the administration of the perfluorocarbon composition relieves the subject's pruritus for 4 hours or more.

In one another embodiment, the administration of the perfluorocarbon composition relieves the subject's pruritus within immediately upon administration to the subject. In another embodiment, the administration of the perfluorocarbon composition relieves the subject's pruritus within 1 minute of the administration. In another embodiment, the administration of the perfluorocarbon composition relieves the subject's pruritus within 2 minute of the administration. In another embodiment, the administration of the perfluorocarbon composition relieves the subject's pruritus within 3 minute of the administration. In yet another embodiment, the administration of the perfluorocarbon composition relieves the subject's pruritus within 5 minute of the administration. In one embodiment, the subject is human.

The subject application also provides for a process of manufacturing the claimed perfluorocarbon gel composition comprising the steps: a) mixing aqueous phase components in a vessel; b) homogenizing the mixture; c) adding perfluorocarbon to the mixture over time during high speed homogenization; and d) obtaining the gel.

In one embodiment, in step a) the aqueous phase components include distilled water, surfactants and/or preservatives. In another embodiment, in step a) the vessel is a glass, polyethylene, PET, or stainless steel vessel.

In one embodiment, in step b) the homogenizer is a rotor stator homogenizer. In another embodiment, in step b) the mixture is homogenized for 4-6 minutes. In another embodiment, in step b) the mixture is homogenized for 5 minutes. In yet another embodiment, in step b) the mixture is homogenized at 10,000-35,000 RPM. In another embodiment, in step c) the perfluorocarbon is added in aliquots or continuously over 10-30 minutes.

The subject application provides for a method of delivering oxygen to a periocular skin of a subject comprising topically administering to a facial area consisting of the periocular skin of the subject the claimed perfluorocarbon composition effective to deliver oxygen to the periocular skin.

The subject application also provides for a method of improving the appearance of a periocular skin of a subject comprising topically administering to a facial area consisting of the periocular skin of the subject the claimed perfluorocarbon composition effective to improve the appearance of the periocular skin.

The subject application also provides for a method of decreasing the Fitzpatrick Wrinkle Assessment Scale score of a subject's skin comprising topically administering to the skin of the subject the claimed perfluorocarbon composition effective to decrease the Fitzpatrick Wrinkle Assessment Scale score.

The subject application also provides for a method of improving the Global Aesthetic Improvement Scale score of a subject's skin comprising topically administering to the skin of the subject the claimed perfluorocarbon composition effective to increase the Global Aesthetic Improvement Scale score.

The subject application provides a method of treating pruritus comprising administering to the skin of a subject afflicted with pruritus an amount of the claimed perfluorocarbon composition effective to treat the pruritus.

The subject application also provides a method of alleviating a symptom of psoriasis comprising administering to the skin of a subject afflicted with psoriasis an amount of the claimed perfluorocarbon composition effective to alleviate the symptom of psoriasis. In one embodiment, the symptom is pruritus.

The subject application also provides the claimed perfluorocarbon composition for use in treating a subject afflicted with pruritus or psoriasis.

The subject application further provides the claimed perfluorocarbon composition comprising an amount of a perfluorocarbon for use in treating pruritus or psoriasis.

The subject application further provides the disclosed perfluorocarbon composition for use in delivering oxygen to the skin of a subject, for use in increasing the firmness of the skin or reducing the appearance of fine lines, wrinkles or scars in a subject, for use in improving the appearance of the skin of a subject, and for use in treating a wound, a burn injury, pruritus, psoriasis, acne or rosacea in a subject suffering therefrom.

It is understood that where a parameter range is provided, all integers within that range, and tenths thereof, are also provided by the invention. For example, “1-90 wt %” includes 1.0 wt %, 1.1 wt %, 1.2 wt %, 1.3 wt %, 1.4 wt % etc. up to 90.0 wt %.

All combinations and sub-combinations of the various elements described herein are within the scope of the invention.

The biochemistry of wound healing and strategies for wound treatment is described Chin et al., (2007) “Biochemistry of Wound Healing in Wound Care Practice” Wound Care Practice, 2^{nd} ed., Best Publishing, AZ., which is hereby incorporated by reference.

Acne treatments are described in section 10, chapter 116, pp 811-813 of The Merck Manual, 17^{th} Edition (1999), Merck Research Laboratories, Whitehouse Station, N.J., U.S.A. which is hereby incorporated by reference.

### Terms

As used herein, and unless stated otherwise, each of the following terms shall have the definition set forth below.

“About” in the context of a numerical value or range means ±10% of the numerical value or range recited or claimed.

“Accelerates healing” as used herein means an increased rate of burn injury/wound repair and healing as compared to the rate of burn injury/wound repair and healing in an untreated control subject.

“Administering to the subject” means the giving of, dispensing of, or application of medicines, drugs, or remedies to a subject to relieve or cure a condition, e.g., a pathological condition. “Topical administration” of a composition as used herein shall mean application of the composition to the skin of a subject. In an embodiment, topical administration of a composition is application of the composition to the epidermis of a subject.

“Ameliorating” a condition or state as used herein shall mean to lessen the symptoms of that condition or state. To “ameliorate” with regard to skin comedones, pustules or papules is to reduce the discomfort caused by comedones, pustules or papules and/or to reduce their appearance and/or physical dimensions.

“Antibacterial agent” means a bactericidal compound such as silver nitrate solution, mafenide acetate, or silver sulfadiazine, or an antibiotic. According to the present invention, antibacterial agents can be present in “Curpon™” products. “Cupron™” products utilize the qualities of copper and binds copper to textile fibers, allowing for the production of woven, knitted and non-woven fabrics containing copper-impregnated fibers with the antimicrobial protection against microorganisms such as bacteria and fungi.

“Biologically active agent” means a substance which has a beneficial or adverse effect on living matters.

“Burn wound” means a wound resulting from a burn injury, which is a first, second or third degree injury caused by thermal heat, radiation, electric or chemical heat, for example as described at page 2434, section 20, chapter 276, of The Merck Manual, 17^{th} Edition (1999), Merck Research Laboratories, Whitehouse Station, N.J., U.S.A.

“Cream” means a liquid or semi-liquid colloid at ambient temperature wherein the dispersed phase is dispersed in a liquid/semi-liquid continuous medium. The cream is more viscous than a liquid but less viscous than a gel. The use of the term “cream” in this application specifically excludes “gel”.

“Effective” as in an amount effective to achieve an end means the quantity of a component that is sufficient to yield a desired therapeutic response with a reasonable benefit/risk ratio of side effects. For example, an amount effective to treat pruritus, without causing unreasonable adverse side effects. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

“Fitzpatrick Wrinkle Assessment Scale” or “FWAS” is a 9-grade scale for assessing the diverse aspects of aging skin. FWAS ranks the depth of the wrinkle (e.g., fine lines or deep wrinkles) and elastosis, the process of increasing the amount of elastic tissue and improving the pliability of the skin. FWAS is commonly used in dermatology to determine the effectiveness of skin care treatments and therapies.

“FnBu” as used herein means perfluoro(n-butylcyclohexane).

“Gel” means a semi-solid or solid colloid (depending on concentration and/or temperature) of a solid/semi-solid and a liquid wherein a liquid dispersed phase is dispersed in a solid/semi-solid continuous medium. Some gels become fluids due to agitation then resume their gel structure when allowed to be undisturbed. Common pharmaceutical gels are solids which when applied and with motion allow the product to become temporarily a liquid phase so it applies smoothly, then becomes tacky then dries. Other gels are semi solid which are a semi-liquid, semi-solid mixture & when applied become tacky then dry. “Hydrogel” means any colloid in which the particles are in the external dispersion phase and water is in the internal dispersed phase.

“Global Aesthetic Improvement Scale” or “CAIS” is another commonly used scale used for assessing changes to skin after treatment is applied. The CAIS rates changes on a scale of one-to-five (1-5), with one (1) being the most improved and five (5) indicating that the appearance has worsened.

“Infection” as used in respect to Propionibacterium acnes means a detrimental colonization of the (host) subject by the Propionibacterium acnes causing an inflammation response in the subject.

“Oxygen tension” or “tissue oxygen tension” is the directly measured local partial pressure of oxygen in a specific tissue.

“Oxygenated perfluorocarbon” is a perfluorocarbon which is carrying oxygen at, for example, saturation or sub-saturation levels.

“Periocular skin” means the skin in the region around the eye, specifically, the skin in the region bounded by the brow superiorly, the infraorbital rim inferiorly, the nose medially and the lateral orbital rim.

“Pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject. The carrier may be liquid or solid and is selected with the planned manner of administration in mind.

“Pharmaceutically active compound” means the compound or compounds that are the active ingredients in a pharmaceutical formulation.

“Promotes alleviation of pain” means a decrease in the subject's experience of pain resulting from a wound or an injury, e.g., a burn injury.

“Sex organ” or “sexual organ” means any of the anatomical parts of the body which are involved in sexual reproduction and constitute the reproductive system in a complex organism. In a preferred embodiment of this invention, the sex organ is the genitalia of the subject. As used herein, the “genitalia” refer to the externally visible sex organs: in males the penis, in females the clitoris and vulva.

“Surfactants” means wetting agents that lower the surface tension of a liquid, allowing easier spreading, and lower the interfacial tension between two liquids. According to one embodiment of the present invention, the surfactants can be Poloxamer 105 (available from BASF Corporation of Mt. Olive, N.J. as Pluronic® L35) or Poloxamer 188 (available from BASF Corporation of Mt. Olive, N.J. as Pluronic® F68) Poloxamer 188 or Poloxamer 407, or a mixture thereof.

“Treating” encompasses, e.g., inducing inhibition, regression, or stasis of, or ameliorating or alleviating a symptom of a condition, e.g., a dermatological condition. “Ameliorating” or “alleviating” a condition or state as used herein shall mean to relieve or lessen the symptoms of that condition or state. “Ameliorate” or “alleviate” with regard to pruritus is to reduce the discomfort or sensation associated with pruritus and/or to reduce tissue damage resulting from or other symptoms associated with pruritus.

“wt %” when referring to the percentage of a component in the claimed composition is percentage of the weight of the component in the composition relative to the total weight of the composition.

The perfluoro(n-butylcyclohexane) (“FnBu”) Compositions

Use of perfluoro(tert-butylcyclohexane) in gel formulations has been suggested for certain uses in U.S. Patent Application Publication No. 2010-0144861, published Jun. 10, 2010. However, since perfluoro(tert-butylcyclohexane) and perfluoro(n-butylcyclohexane) are structurally different, one cannot be simply substituted for another in a formulation. Re-formulation is required.

In one embodiment of the present invention, the FnBu composition is in the form of a gel, a representative gel formulation is shown in Table 1. The PFC gel can be manufactured, e.g., in accordance with the methods disclosed in U.S. Patent Application Publication No. U.S. 2010-0144861, published Jun. 10, 2010, the entirety of which is hereby incorporated by reference into this application.

In another embodiment of the present invention, the FnBu composition is in the form of a cream, representative cream formulations are shown in Tables 2-5. The characteristics of the representative cream formulations is shown in Table 6.

The perfluorocarbon cream provided by this application can contain components from the following list: FnBu, water, cyclopentasiloxane, propanediol, caprylic/capric triglyceride, butylene glycol, glycerin, Butyrospermum parkii (shea butter) dimethicone, cetyl phosphate, stearic acid, Limnanthes alba (meadowfoam) seed oil, glyceryl stearate, PEG-100 sterate, ascorbyl glucoside, Helianthus annuus (sunflower) seed oil unsaponifiables, Persea gratissima (avocado) oil unsaponifiables, Fraxinus excelsior bark extract, Avena sativa (oat) kernel extract, dipotassium glycyrrhizate, niacinamide, palmitoyl oligopeptide, palmitoyl tetrapeptide-7, acrylates/dimethicone copolymer, steareth-20, silanetriol, N-hydroxysuccinimide, chrysin, polyurethane-40, silica, potassium citrate, polysorbate 20, carbomer, disodium EDTA, sodium hydroxide, caprylyl glycol, chlorphenesin, phenoxyethanol, fragrance (parfum), Green 5, aminomethyl propanol, sorbitan olivate, cetearyl olivate, cetyl alcohol, Simmondsia chinensis (jojoba) Seed Oil, dimethicone crosspolymer, phospholipids, tocopheryl acetate, retinyl palmitate, ascorbyl palmitate, Alaria esculenta extract, microcrystalline cellulose, cellulose gum, xanthan gum, sodium phytate, heptyl undecylenate, Olea europaea (olive) fruit oil, hydrogenated olive oil, hydrogenated vegetable oil, Olea europaea (olive) oil unsaponifiables, Crithmum maritimum extract, tetrahexyldecyl ascorbatem, cetearyl alcohol, cetearyl glucoside, sodium citrate, hydrogenated lecithin, sodium oleate, oligopeptide-68, glycine soja (soybean) oil, Pentylene glycol, sodium PCA, isopropyl isostearate, ethylhexylglycerin, hexylene glycol, glucosemine HCl, Pisum sativum (pea) extract, and Bambusa vulgaris leaf/stern extract.

It is known that skin cells need oxygen to regenerate and thrive. Therefore, the PFC composition described herein can be used as a vehicle to topically deliver oxygen to the skin. The PFC composition disclosed herein can increase the oxygen concentration in the treated skin locally as compared to the untreated skin. To further increase oxygen concentration, the PFC composition can be pre-loaded with molecular oxygen. The composition can deliver oxygen to the skin via a diffusion gradient.

The perfluorocarbon compositions and methods described herein may further comprise pharmaceutically acceptable carrier or cosmetic carrier and adjuvant(s) suitable for topical administration. Compositions suitable for topical administration are well known in the pharmaceutical and cosmetic arts. These compositions can be adapted to comprise the oxygenated perfluorocarbon. The composition employed in the methods described herein may also comprise a pharmaceutically acceptable additive.

The multiplicity of configurations may contain additional beneficial biologically active agents which further promote tissue health.

The compositions of this invention may be administered in forms detailed herein. The use of the perfluorocarbon composition may be a component of a combination therapy or an adjunct therapy. The combination therapy can be sequential or simultaneous. The compositions can be administered independently by the same route or by two or more different routes of administration depending on the dosage forms employed.

The dosage of the compounds and compositions administered in treatment will vary depending upon factors such as the pharmacodynamic characteristics of a specific therapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds and compositions may comprise a single compound or mixtures thereof with other compounds. The compounds can be introduced directly into the targeted tissue, using dosage forms well known to those of ordinary skill in the cosmetic and pharmaceutical arts.

The compounds and compositions can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical and cosmetic practices. The compounds can be administered alone but are generally mixed with a pharmaceutically acceptable carrier. This carrier can be a solid or liquid, and the type of carrier is generally chosen based on the type of administration being used. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Techniques and compositions for making dosage forms useful in the present invention are described in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences, Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.). All of the aforementioned publications are incorporated by reference herein.

The PFC compositions may contain the any of the following non-toxic auxiliary substances:

The PFC compositions may contain antibacterial agents which are non-injurious in use, for example, thimerosal, benzalkonium chloride, methyl and propyl paraben, benzyldodecinium bromide, benzyl alcohol, or phenylethanol.

The PFC compositions may also contain buffering ingredients such as sodium acetate, gluconate buffers, phosphates, bicarbonate, citrate, borate, ACES, BES, BICINE, BIS-Tris, BIS-Tris Propane, HEPES, HEPPS, imidazole, MES, MOPS, PIPES, TAPS, TES, and Tricine.

The PFC compositions may also contain a non-toxic pharmaceutical organic carrier, or with a non-toxic pharmaceutical inorganic carrier. Typical of pharmaceutically acceptable carriers are, for example, water, mixtures of water and water-miscible solvents such as lower alkanols or aralkanols, vegetable oils, peanut oil, polyalkylene glycols, petroleum based jelly, ethyl cellulose, ethyl oleate, carboxymethyl-cellulose, polyvinylpyrrolidone, isopropyl myristate and other conventionally employed acceptable carriers.

The PFC compositions may also contain non-toxic emulsifying, preserving, wetting agents, bodying agents, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, thimerosal, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic.

The PFC compositions may also contain surfactants that might be employed include polysorbate surfactants, polyoxyethylene surfactants, phosphonates, saponins and polyethoxylated castor oils, but preferably the polyethoxylated castor oils. These surfactants are commercially available. The polyethoxylated castor oils are sold, for example, by BASF under the trademark Cremaphor.

The PFC compositions may also contain wetting agents commonly used in ophthalmic solutions such as carboxymethylcellulose, hydroxypropyl methylcellulose, glycerin, mannitol, polyvinyl alcohol or hydroxyethylcellulose and the diluting agent may be water, distilled water, sterile water, or artificial tears, wherein the wetting agent is present in an amount of about 0.001% to about 10%.

The formulation of this invention may be varied to include acids and bases to adjust the pH; tonicity imparting agents such as sorbitol, glycerin and dextrose; other viscosity imparting agents such as sodium carboxymethylcellulose, microcrystalline cellulose, polyvinylpyrrolidone, polyvinyl alcohol and other gums; suitable absorption enhancers, such as surfactants, bile acids; stabilizing agents such as antioxidants, like bisulfites and ascorbates; metal chelating agents, such as sodium edetate; and drug solubility enhancers, such as polyethylene glycols. These additional ingredients help make commercial solutions with adequate stability so that they need not be compounded on demand.

Finally, the formulation of this invention can be adjusted so that the PFC composition is the form of a cream, gel, emulsion, pomade, shampoo, conditioner, lotion, liquid, potion, foam, spray, sol, aerosol or similar product, which are suitable for topical application.

Other materials as well as processing techniques and the like are set forth in Part 8 of Remington's Pharmaceutical Sciences, 17th edition, 1985, Mack Publishing Company, Easton, Pa., and International Programme on Chemical Safety (IPCS), which is incorporated herein by reference.

All combinations and sub-combinations of the various elements are within the scope of the invention.

### Use of FnBu Composition for Treatment of Various Dermatological Conditions

The PFC compositions described herein (e.g., PFC gels, cream or lotion) can be used for various dermatological conditions including pruritus relief and for providing faster healing of irritated skin.

The PFC compositions can be used for pruritus relief resulting from insect bites, contact dermatitis, atopic dermatitis, eczema, psoriasis etc. Studies have shown that oxygen may inhibit histamine release that is the cause of itch associated with various conditions. It has been disclosed that an oxygen-glucose deprived environment increases histamine release (Shen, 2007). Therefore, the PFC composition can be used, e.g., for relieving pruritus resulting from various underlying conditions.

PFCs have been suggested to have anti-inflammatory properties. Therefore, the PFC compositions can also treat inflammation associated with various skin conditions described herein. The PFC compositions can also reduce redness, swelling and irritation related to, e.g., insect bites, dermatitis, or psoriasis.

By increasing oxygen concentrations, pruritus and general skin irritation can be alleviated. As an additional benefit, the PFC in the composition can also anesthetize skin similar to the way benzocaine does.

Hydrotherapy with perfluorocarbon compositions described herein can be administered as part of the pruritus, psoriasis or dermatitis treatment protocol.

Also, the perfluorocarbon may be administered with aloe vera. Administration of perfluorocarbon in combination with aloe vera allows for delivery of oxygen to the affiliated tissue, as well as coat endothelial cells and decrease edema.

Lastly, perfluorocarbon compositions disclosed herein can be administered along with an antibacterial agent which would decrease infectious complications.

### Wound and Burns Healing and Scar Prevention and Reduction

As discussed, the PFC compositions described herein have numerous applications. For example, the PFC compositions can be used as a protective wound covering or a topical wound dressing. The wound covering or wound dressing can be used with or incorporated into a bandage. The topical composition wound dressing can be used for an approximately 24 hour period to increase availability of oxygen to the skin surface in wounds such as abrasions, minor lacerations, minor cuts, or minor scalds and burns. The composition can be applied to humans or for veterinary use.

Oxygen is key for healing wounds. Wounds do not heal when oxygen is blocked or decreased (e.g., due to broken capillaries). The topically applied PFC composition creates an oxygen rich environment, increasing oxygen concentration in the affected skin tissue, allowing cells to multiply and heal.

The PFC composition can also be used in treating burn injuries. Extra oxygen in blood promotes angiogenesis, the formation of new capillaries. For severely burned subjects, the PFC composition can not only provide oxygen to oxygen-starved unburned tissue but also promote the establishment of new capillary beds that feed newly grafted skin and burned but salvageable skin. Further, studies have shown that PFCs suppress early postburn lipid peroxidation and increases resistance of red blood cells to oxidative hemolysis (Bekyarova, 1997).

In addition to promoting healing of wounds and burns, the PFC compositionl can also prevent scarring. Scars are created when there is not enough oxygen for the skin to correctly heal. Accordingly, increasing oxygen concentrations in the tissue can reduce the appearance of scars.

Therefore, the PFC composition can also prevent scarring by quickly healing minor wounds and reduce the appearance of scars by oxygenating the skin tissue and activating the skin regenerative function.

Similarly, the PFC composition can also be used for topical application after procedures causing tissue damage. For example, the PFC composition can be applied to post-surgery incisions to promote faster healing. Capillaries ultimately oxygenate the cells/tissues. After an injury (which includes surgical incisions), it's the capillaries that are damaged, making them incapable of carrying fluid to and from the damaged tissues. The result is swelling and inflammation.

Increased oxygen levels promote angiogenesis, the growth of new capillaries and the repair of damaged capillaries. Thus, oxygen would accelerate healing of the capillaries and fluid could then again be removed. The PFC composition would also oxygenate the tissues at the same time. When swelling is reduced, the pain caused by inflammation is also reduced. It is envisioned that any medical procedure which causes tissue injury could potentially benefit, e.g., pulling teeth, incisions, etc.

In another example, the PFC composition can be applied post-cosmetic surgery (e.g, post-microdermabrasion or chemical facial peels), both for the soothing effect as well as the acceleration of recovery. Since these procedures literally abrade/remove the top layers of the dermis, the PFC composition can then promotes cell turnover and repair, which should be accelerated by the topical use.

Similarly, the composition can be used to treat burns resulting from radiation in the same way that it treats burns in general as previously discussed.

The PFC composition can be a component of a combination therapy or an adjunct therapy. For example, the composition can be administered with or without hyperbaric or supplemental oxygen. In one embodiment, the subject can be administered the PFC composition disclosed herein in combination with supplemental oxygen. In another embodiment, the PFC composition can be administered in combination with the subject's own white blood cells, increasing the efficacy of the treatment.

### Anti-Aging Cosmetic Use

The PFC compositions described herein (e.g., a perfluorocarbon cream or lotion) can be used as a cosmetic agent to improve the overall appearance of the skin and promote anti-aging, especially in the periocular skin. The PFC composition can be used for reducing skin imperfections such as fine lines, wrinkles, puffiness, dark (under-eye) circles, bags or dark blemishes around the eye. The PFC composition can also be used for the promotion of skin firmness.

Oxygen levels in the skin decrease with age, making the appearance of fine lines and wrinkles more noticeable. A lack of oxygen at the cellular level can cause skin to age prematurely, increasing the appearance of fine lines and age spots, making skin look dry and dull. Applying an oxygen-rich perfluorocarbon composition (e.g., a perfluorocarbon cream) to the skin can enhance oxygen levels in the skin, promote cell turnover and repair, reduce and/or prevent fine lines and wrinkles, thus improving overall appearance and feel of the skin.

In addition, oxygen can inhibit the destructive enzyme collagenase which breaks down collagen. Collagen is one of the structural substances that supports the skin's surface. By supporting collagen production (by inhibiting collagenase through higher oxygen levels), the skin can be firmer and look more youthful.

Therefore, the PFC composition described herein can diminish fine lines and wrinkles by using oxygen to activate the skin regenerative functions and collagen production. Moreover, the PFC composition can increase the firmness and elasticity of the skin by activating collagen and elastin creation.

Yet another cosmetic use for the PFC composition disclosed herein is the reduction of cellulite. By topically applying the PFC composition in combination with caffeine and optionally dimethyl sulfoxide (DMSO), cellulite can be reduced.

The PFC composition can be a component of a combination therapy/treatment or an adjunct therapy/treatment. For example, the PFC cream can be administered in combination with another agent, e.g., a moisturizer, to improve skin appearance and/or improve skin health, or an additive capable of providing sun protection or boost sun protection factor (SPF).

### Treatment of Acne and Rosacea

The PFC composition can also be used to treat skin infirmities such as acne or rosacea. Specially, the PFC composition can prevent, heal and eliminate acne, providing clear and break-out free skin.

Acne can be caused by an anaerobic bacterium infection as well as the inflammatory reaction caused by the release of the bacteria's toxins. Anaerobic bacteria are intolerant of oxygen, replicating at low oxidation-reduction potential sites. Since Propionibacterium acnes is an anaerobic bacterium, it thrives in an environment devoid of oxygen. The addition of oxygen to an anaerobic infection helps to kill the bacteria and improve the dermatological condition called acne. The PFC composition disclosed herein is able to carry a more oxygen than hemoglobin. The PFC composition is able to provide this oxygen through diffusion to an area of low oxygen concentration, such as an anaerobic infection.

Anaerobic bacteria are more susceptible to the effects of oxygen than the more common aerobic bacteria. The PFC composition when applied topically provides increased local oxygen to the acne lesions and helps eradicate Propionibacterium acnes and thus ameliorates the acne.

The introduction of supplemental topical oxygen (in an oxygenated perfluorocarbon or via diffusion through PFC) to a patient who has acne enables the intensity and number of lesions to be eradicated more efficiently than current therapeutic regiments. It helps decrease the extent, duration, super infections and complications (such as scarring) from acne.

Moreover, if large pores are a contributing factor to acne and blemishes, by providing an oxygen-rich environment to the pores, breakouts can be prevented by keeping the pores open and clean. The PFC composition therefore provides increased oxygen to the tissues, a healthy environment is created for cells, allowing them to multiply and thrive.

The application of the topical form of perfluoro(n-butylcyclohexane) in a cream, gel, pomade, shampoo, conditioner, lotion, liquid, potion, foam, or similar product, or in combination with a topical antibiotic, or topical acne product such as retinoid, benzoyl peroxide, peroxide, isotretionoin, etc. to the inflamed and infected area enhances the eradication and prevention of the harmful effects of Propionibacterium acnes. In addition, the PFC composition helps prevent, ameliorate and eradicate superinfections and some of the complications (comedones, pustules, papules, etc.) that acne causes.

Also the PFC composition can eliminate and/or reduce redness and pustules associated with rosacea breakouts. For this indication, the same principles described for acne and other uses apply. The PFC composition increases oxygen levels in the face and should be particularly effective because the capillary bed feeding the face is so vast and they are located very close to the surface of the skin. In addition, rejuvenation and healing mechanism described previously is also applicable.

### Enhancement of Sexual Function

The PFC composition can also be used for enhancing sexual function. Specifically, the PFC composition can be topically used for increasing oxygen delivery to the sex organ of a subject for enhancement of male and female sexual function.

The PFC composition provides to the sex organ an oxygen-rich environment and thus improves sexual response time, the frequency of erections, and the duration of response. Specifically, the PFC composition can be applied topically to the sex organ and absorbed into local circulation, causing trabecular smooth muscles to relax, which is the mechanism leading to an erection.

### Other Indications and Uses

Other indications and uses are summarized as follows:
Canker Sores: The PFC composition can be used for reducing the time it takes to cure canker sores. Oxygen is known to help the immune system fight bacteria and infections. By increasing oxygen concentrations, the body's immune system would be able to fight infections better.
Cavities: The PFC composition can be used in a cavity fighting mouthwash or toothpaste. At night, humans salivate less and therefore do not wash away food particles and harmful bacteria. These bacteria can make their ATP aerobically, but they switch to fermentation if there is no O₂ available. It is this fermentation that lowers the pH on the teeth and cases demineralization and decay. By increasing oxygen, the PFC composition can prevent the fermentation process from taking place.
Decubitus Ulcer: The PFC composition can also be used in the treatment of decubitus ulcers, more commonly known as besores.
   By packing the wound with gauze or other material containing the PFC composition or by coating the large surface area of these types of wounds with the PFC composition, the composition can accelerate healing of the wound from the inside out.
Diabetic Foot Care: The PFC composition can be used in the treatment of the diabetic foot by providing an oxygen-rich environment to the diabetic foot as well as adding a protective barrier which may be provided by the surfactant, thus keeping the skin of the diabetic foot soft, preventing it from becoming dry and then cracking, which often leads to more serious foot wounds and infections.
Gas Gangrene: The PFC composition can be used for fighting deadly infections caused by gas gangrene. Gas-producing organisms (such as those that cause toxic shock syndrome and gas gangrene and botulism) cause their damage by releasing toxic gases into the tissues/body. These organisms are anaerobic. Therefore, by providing an oxygen-rich environment, the anaerobic organisms would be destroyed by oxygen.
   As an additional benefit, the PFC composition can absorb the toxic gases released from the organisms.
Hemorrhoids: PFC composition disclosed herein can be used in the treatment of hemorrhoids, specifically, in relieving inflammation, reducing swelling and associated pain in addition to reducing incidence of necrosis. Hemorrhoids are varicose veins and as such, their blood supply is compromised. Application of an oxygen-enhancing composition will bring needed oxygen to the area, which will prevent necrosis of the tissues. Since inflammation is a response to tissue injury, and in this case, the injury is caused by limited oxygen supply, replenishing the oxygen supply would reduce the inflammation, thereby reducing the swelling and associated pain.
Muscle Pain/Aching Muscle: The PFC composition can be used for the treatment of muscle pain. The composition can be applied to the muscles to provide oxygen before, during, or after strenuous exercise. In one embodiment, the composition can be combined with an ingredient which provides heat to the muscles, such as camphor or eucalyptus.
   The composition can also be used for speeding up the healing process of muscle tears. Strenuous activity creates small tears in muscle tissue. The Healing of these tears increases muscle mass. The PFC composition will increase oxygen tension in the muscle and hence, speed up the healing process.
Nocturnal Leg Cramps: PFC composition disclosed herein can be used in the treatment of nocturnal leg cramps by increasing oxygen levels in the lower leg during sleep.
   Nocturnal leg cramps affect nearly 70% of the population. Various causes include dehydration, electrolyte imbalance and decreased oxygen to the limbs (also caused by various factors). Even when cramping is caused by dehydration/electrolyte imbalance, it is ultimately the decrease in oxygen, secondary possibly to the root cause that causes the muscles to cramp. Therefore, the PFC composition can be used in the treatment of nocturnal leg cramps by increasing oxygen levels in the lower leg during sleep.
Shampoo, Conditioner, Dandruff or Hair Loss Treatment: The PFC composition can also be incorporated into hair products such as shampoo and conditioners, enhancing oxygen concentration when applied. Pollutants in the air are known to make hair drab and dull. By increasing oxygen to the hair, the hair would be revitalized.
   The composition would also moisturize hair and protects it from heat when styling. The composition can also reduce frizz in hair.
   At the same time, oxygenating and moisturizing the scalp creates a healthy and hydrated scalp. Having a healthy and hydrated scalp would reduce the likelihood of dandruff and therefore, of fungal colonization of the scalp that is often caused by dandruff.
   Moreover, the PFC composition can aid in hair growth. The PFC composition can increase generation of capillaries that feed the scalp, thereby increasing blood flow and oxygenation to hair follicles.
Skin Graft: The PFC composition can also accelerate skin graft uptake and increase in skin graft survival.
   For skin grafts, it is critical to restore the circulation to the grafted tissues as soon as possible. As discussed previously, oxygen promote angiogenesis, the growth of new capillaries and the repair of damaged capillaries. Again, it is the capillaries which feed the tissues by carrying fluid to and from the tissues.
   By topically applying the PFC composition and promoting angiogenesis, the composition can promote re-epithelialization, healing and graft acceptance by bringing additional oxygen to the epithelial cells.

This invention will be better understood by reference to the Experimental Details which follow, but those skilled in the art will readily appreciate that the specific experiments detailed are only illustrative of the invention as described more fully in the claims which follow thereafter.

### Experimental Details

### Example 1

### Representative FnBu Compositions

TABLE 1
Representative FnBu Gel

| | | |
|---|---|---|
| Component | grams | Wt % |

| | | | |
|---|---|---|---|
| Vitamin E | 0.017 | g | 0.03 (300 ppm) |
| Pluronic ® L35 | 1.4 | g | 2.43 |
| Pluronic ® F68 | 0.15 | g | 0.26 |
| Water | 6.0 | g | 10.42 |
| perfluoro(n- | 50 | g | 86.86 |
| butylcyclohexane) | | | |

TABLE 2
Representative FnBu Composition.

| CAS # | Trade Name | INCI Name | Wt. % | Breakdown |
|---|---|---|---|---|
| N/A | perfluoro(n- | perfluoro(n- | 15.00-90.00% | 97% |
| | butylcyclohexane) | butylcyclohexane) | | |
| 7732-18-5 | Deionized Water | Water (Aqua) | 15.00-90.00% | 100% |
| 76050-42-5 | Carbopol Ultrez-10 | Carbomer | 0-1.00% | 100% |
| 504-63-2 | Zemea | Propanediol | 1-10.00% | 100% |
| 139-33-3 | Dissolvine Na-2-P | Disodium EDTA | 0-1.00% | 100% |
| 91770-40-0 | Avocadin (HU-25) | Persea gratissima | 0-1.00% | 100% |
| | | (Avocado) Oil | | |
| | | Unsaponifiables | | |
| 68920-03-6 | Shea Butter Refined | Butyrospermum parkii | 1-10.00% | 100% |
| | or “Ultra Refined” | (Shea Butter) | | |
| 57-11-4 | Stearic Acid | Stearic Acid | 1-10.00% | 100% |
| 3539-43-3 | Amphisol A | Cetyl Phosphate | 1-10.00% | 100% |
| 31566-31-1 | Simulsol 165 | Glyceryl Stearate | 1-10.00% | 50% |
| 9004-99-3 | | PEG-100 Stearate | | 50% |
| 63148-62-9 | Dow Corning 200 | Dimethicone | 1-10.00% | 100% |
| | Fluid 350 CST | | | |
| 541-02-6 | Volasil 995 | Cyclopentasiloxane | 1-10.00% | 99% |
| 65381-09-1 | Myritol312 | Caprylic/Capric | 1-10.00% | 100% |
| | | Triglyceride | | |
| 541-02-6 | KP-545 | Cyclopentasiloxane | 1-10.00% | 70% |
| N/A | | Acrylates/Dimethicone | | 30% |
| | | Copolymer | | |
| 8001-21-6 | Soline | Helianthus annuus | 0-1.00% | 100% |
| | | (Sunflower) Seed Oil | | |
| | | Unsaponifiables | | |
| N/A | Botanol MO | Limnanthes alba | 1-10.00% | 100% |
| | | (Meadowfoam) Seed Oil | | |
| 1310-73-2 | Sodium Hydroxide, | Sodium Hydroxide | 0-1.00% | 100% |
| | Pellets, NF | | | |
| 122-99-6 | Mikrokill Cos | Phenoxyethanol | 1-10.00% | 66% |
| 1117-86-8 | | Caprylyl Glycol | | 15% |
| 104-29-0 | | Chlorphenesin | | 19% |
| 68797-35-3 | ARG-DPG | Dipotassium Glycyrrhizate | 0-1.00% | 100% |
| 129499-78-1 | AA2G | Ascorbyl Glucoside | 1-10.00% | 100% |
| 7732-18-5 | Drago-Calm 674463 | Water (Aqua) | 0-1.00% | 49.5% |
| 56-81-5 | | Glycerin | | 49.5% |
| 84012-26-0 | | Avena sativa (Oat) Kernel Extract | | 1% |
| 107-88-0 | Cytobiol Lumin-Eye | Butylene Glycol | 1-10.00% | 37% |
| 7732-18-5 | | Water (Aqua) | | 37% |
| 98-92-0 | | Niacinamide | | 18% |
| 84625-28-5 | | Fraxinus excelsior Bark Extract | | 6.2% |
| N/A | | Silanetriol | | 0.4% |
| 866-84-2 | | Potassium Citrate | | 0.4% |
| 7732-18-5 | Haloxyl | Water (Aqua) | 1-10.00% | 80.7750% |
| 56-81-5 | | Glycerin | | 15% |
| 9005-00-9 | | Steareth-20 | | 4% |
| 6066-82-6 | | N-Hydroxysuccinimide | | 0.2% |
| 480-40-0 | | Chrysin | | 0.01% |
| 147732-56-7 | | Palmitoyl Oligopeptide | | 0.01% |
| 221227-05-0 | | Palmitoyl Tetrapeptide-7 | | 0.005% |
| 56-81-5 | Matrixyl 3000 | Glycerin | 1-10.00% | 53.4850% |
| 7732-18-5 | | Water (Aqua) | | 25% |
| 107-88-0 | | Butylene Glycol | | 20% |
| 76050-42-5 | | Carbomer | | 1% |
| 9005-64-5 | | Polysorbate 20 | | 0.5% |
| 147732-56-7 | | Palmitoyl Oligopeptide | | 0.01% |
| 221227-05-0 | | Palmitoyl Tetrapeptide-7 | | 0.005% |
| N/A | Fragrance - “Silky | Fragrance (Parfum) | 0-1.00% | 100% |
| | Skin” - #6110985 | | | |
| 112945-52-5 | ChronoSphere | Silica | 1-10.00% | 3% |
| N/A | Opticals | Polyurethane-40 | | 5% |
| 4403-90-1 | Brite | Green 5 | | 92% |

TABLE 3
Representative FnBu Composition.

| CAS # | Trade Name | INCI Name | Wt. % | Break-down |
|---|---|---|---|---|
| N/A | perfluoro(n- | perfluoro(n- | 15.00-90.00% | 97% |
| | butylcyclohexane) | butylcyclohexane) | | |
| 7732-18-5 | Deionized Water | Water (Aqua) | 15.00-90.00% | 100% |
| 76050-42-5 | Carbopol Ultrez-10 | Carbomer | 0-1.00% | 100% |
| 504-63-2 | Zemea | Propanediol | 1-10.00% | 100% |
| 139-33-3 | Dissolvine Na-2-P | Disodium EDTA | 0-1.00% | 100% |
| 91770-40-0 | Avocadin (HU-25) | Persea gratissima | 0-1.00% | 100% |
| | | (Avocado) Oil | | |
| | | Unsaponifiables | | |
| 68920-03-6 | Shea Butter Refined | Butyrospermum parkii | 1-10.00% | 100% |
| | or “Ultra Refined” | (Shea Butter) | | |
| 57-11-4 | Stearic Acid | Stearic Acid | 1-10.00% | 100% |
| 3539-43-3 | Amphisol A | Cetyl Phosphate | 1-10.00% | 100% |
| 31566-31-1 | Simulsol 165 | Glyceryl Stearate | 1-10.00% | 50% |
| 9004-99-3 | | PEG-100 Stearate | | 50% |
| 63148-62-9 | Dow Corning 200 | Dimethicone | 1-10.00% | 100% |
| | Fluid 350 CST | | | |
| 541-02-6 | Volasil 995 | Cyclopentasiloxane | 1-10.00% | 99% |
| 65381-09-1 | Myritol312 | Caprylic/Capric | 1-10.00% | 100% |
| | | Triglyceride | | |
| 541-02-6 | KP-545 | Cyclopentasiloxane | 1-10.00% | 70% |
| N/A | | Acrylates/Dimethicone | | 30% |
| | | Copolymer | | |
| 8001-21-6 | Soline | Helianthus annum | 0-1.00% | 100% |
| | | (Sunflower) Seed Oil | | |
| | | Unsaponifiables | | |
| N/A | Botanol MO | Limnanthes alba | 1-10.00% | 100% |
| | | (Meadowfoam) Seed Oil | | |
| 1310-73-2 | Sodium Hydroxide, | Sodium Hydroxide | 0-1.00% | 100% |
| | Pellets, NF | | | |
| 122-99-6 | Mikrokill Cos | Phenoxyethanol | 1-10.00% | 66% |
| 1117-86-8 | | Caprylyl Glycol | | 15% |
| 104-29-0 | | Chlorphenesin | | 19% |
| 68797-35-3 | ARG-DPG | Dipotassium Glycyrrhizate | 0-1.00% | 100% |
| 129499-78-1 | AA2G | Ascorbyl Glucoside | 1-10.00% | 100% |
| 7732-18-5 | Drago-Calm 674463 | Water (Aqua) | 0-1.00% | 49.5% |
| 56-81-5 | | Glycerin | | 49.5% |
| 84012-26-0 | | Avena sativa (Oat) Kernel Extract | | 1% |
| 107-88-0 | Cytobiol Lumin-Eye | Butylene Glycol | 1-10.00% | 37% |
| 7732-18-5 | | Water (Aqua) | | 37% |
| 98-92-0 | | Niacinamide | | 18% |
| 84625-28-5 | | Fraxinus excelsior Bark Extract | | 6.2% |
| N/A | | Silanetriol | | 0.4% |
| 866-84-2 | | Potassium Citrate | | 0.4% |
| 7732-18-5 | Haloxyl | Water (Aqua) | 1-10.00% | 80.7750% |
| 56-81-5 | | Glycerin | | 15% |
| 9005-00-9 | | Steareth-20 | | 4% |
| 6066-82-6 | | N-Hydroxysuccinimide | | 0.2% |
| 480-40-0 | | Chrysin | | 0.01% |
| 147732-56-7 | | Palmitoyl Oligopeptide | | 0.01% |
| 221227-05-0 | | Palmitoyl Tetrapeptide-7 | | 0.005% |
| 56-81-5 | Matrixyl 3000 | Glycerin | 1-10.00% | 53.4850% |
| 7732-18-5 | | Water (Aqua) | | 25% |
| 107-88-0 | | Butylene Glycol | | 20% |
| 76050-42-5 | | Carbomer | | 1% |
| 9005-64-5 | | Polysorbate 20 | | 0.5% |
| 147732-56-7 | | Palmitoyl Oligopeptide | | 0.01% |
| 221227-05-0 | | Palmitoyl Tetrapeptide-7 | | 0.005% |
| N/A | Fragrance - “Silky | Fragrance (Parfum) | 0-1.00% | 100% |
| | Skin” - #6110985 | | | |
| 112945-52-5 | ChronoSphere | Silica | 1-10.00% | 3% |
| N/A | Opticals Brite | Polyurethane-40 | | 5% |
| 4403-90-1 | | Green 5 | | 92% |
| 124-68-5 | AMP Ultra PC 2000 | Aminomethyl Propanol | 0-1.00% | 100% |
| 223706-40-9 | Olivem 1000 | Sorbitan Olivate | 0-10.00% | 40% |
| INCI ID 16267 | | Cetearyl Olivate | | 60% |
| 36653-82-4 | Lanette 16 | Cetyl Alcohol | 0-10.00% | 100% |
| 65381-09-01 | Lipohate GC | Caprylic/Capric | 0-10.00% | 100% |
| | | Triglyceride | | |
| 61789-91-1 | Jojoba Oil Colorless | Simmondsia chinensis | 0-10.00% | 100% |
| | | (Jojoba) Seed Oil | | |
| 8024-32-6 | Avocado Oil | Persea gratissima | 0-10.00% | 100% |
| | | (Avocado) Oil | | |
| 63148-62-9 | Dimethisil DM-350 | Dimethicone | 0-10.00% | 100% |
| 7732-18-5 | Brookosome ACE | Water (Aqua) | 0-1.00% | 84.91% |
| 123465-35-0 | | Phospholipids | | 13.50% |
| 7695-91-2 | | Tocopheryl Acetate | | 0.07% |
| 79-81-2 | | Retinyl Palmitate | | 0.3% |
| 137-66-6 | | Ascorbyl Palmitate | | 0.02% |
| 65381-09-1 | Kalpariane | Caprylic/Capric Triglyceride | 0-10.00% | 95% |
| N/A | | Alaria esculenta extract | | 5% |
| 63148-62-9 | Dow Corning 9041 | Dimethicone | 0-10.00% | 70% |
| 213629-14 -2 | Silicone Elastomer | Dimethicone Crosspolymer | | 30% |
| | Blend | | | |
| 9004-34-6 | Avicel PC-591 | Microcrystaline Cellulose | 0-10.00% | 85% |
| 9004-32-4 | | Cellulose Gum | | 15% |
| 56-81-5 | Glycerine 99.7% USP | Glycerine | 0-10.00% | 99.7% |
| 7732-18-5 | | Water (Aqua) | | 0.3% |
| 11138-68-2 | Keltrol CG | Xanthan Gum | 0-1.00% | 100% |
| 7732-18-5 | Dermofeel PA-3 | Water (Aqua) | 0-1.00% | 50% |
| 34367-89-0 | | sodium phytate | | 50% |
| 68141-27-5 | LexFeel Natural | Heptyl undecylenate | 0-10.00% | 100% |
| 8001-25-0 | Oilwax | Olea europaea (Olive) | 0-10.00% | 65% |
| | | Fruit Oil | | |
| INCI Monos ap | | Hydrogenated Olive Oil | | 27.5% |
| 156798-12-8 | | Olea europaea (Olive) Oil | | 7.5% |
| | | Unsaponifiables | | |
| 67762-27-0 | Lanette O | Cetearyl Alcohol | 0-10.00% | 100% |
| 65381-09-1 | Arophira | Caprylic/Capric | 0-1.00% | 75% |
| | | triglyceride | | |
| 68334-28-1 | | Hydrogenated Vegetable Oil | | 20% |
| 89997-98-8 | | Crithmum maritimum extract | | 5% |
| 183476-82-6 | BV-OSC | Tetrahexyldecyl ascorbate | 0-10.00% | 100% |
| 67762-27-0 | Montanov 68 | Cetearyl Alcohol | 0-10.00% | 80% |
| INCI ID 8028 | | Cetearyl Glucoside | | 20% |
| 6132-04-3 | Sodium Citrate USP | Sodium Citrate | 0-1.00% | 100% |
| 7732-18-5 | B-White | Water (Aqua) | 0-10.00% | 64.5522% |
| 107-88-0 | | Butylene Glycol | | 35% |
| 92128-87-5 | | Hydrogenated Lecithin | | 0.02% |
| 143-19-1 | | Sodium Oleate | | 0.002% |
| 139-33-3 | | Disodium EDTA | | 0.0008% |
| INCI ID 24888 | | Oligopeptide-68 | | 0.95% |
| 56-81-5 | | Glycerin | | 0.2% |
| 8001-22-7 | | Glycine Soja (Soybean) Oil | | 0.02% |
| 5343-92-0 | Hydrolite-5 #516751 | Pentylene glycol | 0-5.00% | 100.00% |
| 28874-51-3 | Ajidew NL-50 | Sodium PCA | 0-5.00% | 50.00% |
| 7732-18-5 | | Water (Aqua) | | 50.00% |
| 31478-84-9 | Schercemol 318 Ester | isopropyl isostearate | 0-10.00% | 100.00% |
| 122-99-6 | Botanistat PF-64 | phenoxyethanol | 0.5.00% | 50.00% |
| 1117-86-8 | | caprylyl glycol | | 25.00% |
| 70445-33-9 | | ethylhexylglycerin | | 12.50% |
| 107-41-5 | | hexylene glycol | | 12.50% |
| 7732-18-5 | Derm SRC - PF | Water (Aqua) | 0-2.00% | 59.00% |
| 66-84-2 | | glucosemine HCl | | 20.00% |
| 90082-41-0 | | Pisum sativum (Pea) extract | | 12.00% |
| 91771-33-4 | | Bambusa vulgaris leaf/stern | | 9.00% |
| | | extract | | |

TABLE 4
Representative FnBu Compositions.

| Trade Name | INCI Name | Range | 1 | 2 | 3 | 4 | 5 |
|---|---|---|---|---|---|---|---|
| perfluoro(n- | perfluoro(n- | 15%-50% | 25.50% | 40.00% | 46.70% | 15.00% | 45.00% |
| butylcyclohexane) | butylcyclohexane) | | | | | | |
| Deionized Water | Water (Aqua) | 15%-50% | 35.50% | 20.00% | 20.50% | 40.00% | 18.40% |
| Carbopol Ultrez-10 | Carbomer | 0.01%-2% | 0.35% | 0.60% | 0.55% | 0.30% | 0.70% |
| Zemea | Propanediol | 1%-10% | 5.50% | 5.03% | 4.50% | 4.00% | 2.00% |
| Dissolvine Na-2-P | Disodium EDTA | 0.01%-2% | 0.15% | 0.02% | 0.14% | 0.10% | 0.16% |
| Avocadin (HU-25) | Persea gratissima | 0.01%-2% | 0.65% | 0.80% | 0.70% | 0.10% | 0.80% |
| | (Avocado) Oil | | | | | | |
| | Unsaponifiables | | | | | | |
| Shea Butter Refined | Butyrospermum parkii | 0.5%-5% | 3.80% | 2.50% | 2.00% | 4.00% | 1.50% |
| or “Ultra Refined” | (Shea Butter) | | | | | | |
| Stearic Acid | Stearic Acid | 0.5%-5% | 1.10% | 1.80% | 1.50% | 2.00% | 2.50% |
| Amphisol A | Cetyl Phosphate | 0.5%-5% | 1.50% | 1.70% | 2.10% | 2.00% | 2.50% |
| Simulsol 165 | Glyceryl Stearate | 0.5%-5% | 2.50% | 3.50% | 1.75% | 2.00% | 1.50% |
| | PEG-100 Stearate | | | | | | |
| Dow Corning 200 | Dimethicone | 0.5%-5% | 2.40% | 1.70% | 1.35% | 3.00% | 1.50% |
| Fluid 350 CST | | | | | | | |
| Volasil 995 | Cyclopentasiloxane | 0.5%-5% | 2.50% | 3.50% | 1.50% | 2.70% | 2.50% |
| Myritol 312 | Caprylic/Capric | 0.5%-5% | 1.80% | 1.50% | 1.45% | 4.00% | 1.00% |
| | Triglyceride | | | | | | |
| KP-545 | Cyclopentasiloxane | 0.5%-5% | 2.50% | 1.50% | 1.80% | 3.00% | 2.94% |
| | Acrylates/Dimethicone | | | | | | |
| | Copolymer | | | | | | |
| Soline | Helianthus annuus | 0.01%-2% | 0.35% | 0.34% | 0.33% | 0.30% | 0.10% |
| | (Sunflower) Seed Oil | | | | | | |
| | Unsaponifiables | | | | | | |
| Botanol MO | Limnanthes alba | 0.5%-5% | 1.80% | 1.30% | 1.70% | 1.50% | 2.00% |
| | (Meadowfoam) Seed | | | | | | |
| | Oil | | | | | | |
| Sodium Hydroxide, | Sodium Hydroxide | 0.1%-5% | 0.37% | 0.88% | 0.76% | 0.35% | 2.00% |
| Pellets, NF | | | | | | | |
| Mikrokill Cos | Phenoxyethanol | 0.1%-5% | 1.40% | 1.20% | 1.10% | 1.80% | 3.00% |
| | Caprylyl Glycol | | | | | | |
| | Chlorphenesin | | | | | | |
| ARG-DPG | Dipotassium | 0.01%-2% | 0.09% | 0.05% | 0.08% | 0.10% | 0.20% |
| | Glycyrrhizate | | | | | | |
| AA2G | Ascorbyl Glucoside | 0.1%-5% | 2.10% | 1.25% | 1.90% | 1.50% | 1.00% |
| Drago-Calm 674463 | Water (Aqua); | 0.01%-2% | 0.12% | 0.15% | 0.35% | 0.30% | 0.10% |
| | Glycerin; Avena sativa | | | | | | |
| | (Oat) Kernel Extract | | | | | | |
| Cytobiol Lumin-Eye | Butylene Glycol; | 1-10% | 3.20% | 5.00% | 3.70% | 2.00% | 2.20% |
| | Water (Aqua); | | | | | | |
| | Niacinamide; Fraxinus | | | | | | |
| | excelsior Bark Extract; | | | | | | |
| | Silanetriol; Potassium | | | | | | |
| | Citrate | | | | | | |
| Haloxyl | Water (Aqua); | 0.1%-5% | 1.80% | 1.25% | 1.40% | 4.00% | 1.55% |
| | Glycerin; Steareth-20; | | | | | | |
| | N- | | | | | | |
| | Hydroxysuccinimide; | | | | | | |
| | Chrysin; Palmitoyl | | | | | | |
| | Oligopeptide; | | | | | | |
| | Palmitoyl | | | | | | |
| | Tetrapeptide-7 | | | | | | |
| Matrixyl 3000 | Glycerin; Water | 0.1%-5% | 1.50% | 1.60% | 1.00% | 4.00% | 2.25% |
| | (Aqua); Butylene | | | | | | |
| | Glycol; Carbomer | | | | | | |
| | Polysorbate 20; | | | | | | |
| | Palmitoyl | | | | | | |
| | Oligopeptide; | | | | | | |
| | Palmitoyl | | | | | | |
| | Tetrapeptide-7 | | | | | | |
| Fragrance - “Silky | Fragrance (Parfum) | 0.01%-2% | 0.02% | 0.08% | 0.04% | 0.95% | 0.10% |
| Skin” - #6110985 | | | | | | | |
| ChronoSphere | Silica; Polyurethane- | 15%-50% | 1.50% | 2.75% | 1.10% | 1.00% | 2.50% |
| Opticals Brite | 40; Green 5 | | | | | | |

| Trade Name | INCI Name | 6 | 7 | 8 | 9 | 10 | 11 |
|---|---|---|---|---|---|---|---|
| perfluoro(n- | perfluoro(n- | 30.50% | 20.00% | 35.00% | 25.00% | 30.00% | 18.00% |
| butylcyclohexane) | butylcyclohexane) | | | | | | |
| Deionized Water | Water (Aqua) | 26.30% | 36.20% | 26.30% | 34.00% | 27.50% | 36.03% |
| Carbopol Ultrez-10 | Carbomer | 0.45% | 0.50% | 0.80% | 0.10% | 0.20% | 0.90% |
| Zemea | Propanediol | 6.50% | 3.00% | 2.50% | 5.00% | 3.50% | 9.00% |
| Dissolvine Na-2-P | Disodium EDTA | 0.20% | 1.00% | 0.08% | 0.03% | 0.05% | 0.50% |
| Avocadin (HU-25) | Persea gratissima | 0.60% | 0.90% | 0.85% | 0.25% | 0.75% | 0.50% |
| | (Avocado) Oil | | | | | | |
| | Unsaponifiables | | | | | | |
| Shea Butter Refined | Butyrospermum parkii | 4.00% | 1.00% | 3.00% | 3.20% | 3.50% | 4.50% |
| or “Ultra Refined” | (Shea Butter) | | | | | | |
| Stearic Acid | Stearic Acid | 1.05% | 3.00% | 1.90% | 1.00% | 2.20% | 1.10% |
| Amphisol A | Cetyl Phosphate | 4.00% | 2.25% | 1.90% | 1.00% | 2.30% | 0.70% |
| Simulsol 165 | Glyceryl Stearate | 2.80% | 3.00% | 3.25% | 4.80% | 4.00% | 3.30% |
| | PEG-100 Stearate | | | | | | |
| Dow Corning 200 | Dimethicone | 2.00% | 1.00% | 1.25% | 1.75% | 1.30% | 1.50% |
| Fluid 350 CST | | | | | | | |
| Volasil 995 | Cyclopentasiloxane | 5.00% | 3.00% | 2.00% | 2.00% | 1.10% | 4.00% |
| Myritol 312 | Caprylic/Capric | 1.20% | 2.00% | 1.75% | 3.00% | 3.25% | 2.00% |
| | Triglyceride | | | | | | |
| KP-545 | Cyclopentasiloxane | 2.75% | 1.00% | 1.75% | 5.00% | 2.00% | 3.50% |
| | Acrylates/Dimethicone | | | | | | |
| | Copolymer | | | | | | |
| Soline | Helianthus annuus | 0.04% | 0.50% | 0.25% | 0.20% | 0.28% | 0.10% |
| | (Sunflower) Seed Oil | | | | | | |
| | Unsaponifiables | | | | | | |
| Botanol MO | Limnanthes alba | 1.90% | 1.75% | 1.40% | 1.40% | 1.60% | 1.00% |
| | (Meadowfoam) Seed | | | | | | |
| | Oil | | | | | | |
| Sodium Hydroxide, | Sodium Hydroxide | 0.41% | 0.75% | 0.46% | 0.50% | 0.70% | 0.48% |
| Pellets, NF | | | | | | | |
| Mikrokill Cos | Phenoxyethanol | 1.50% | 2.10% | 1.30% | 2.00% | 1.00% | 2.20% |
| | Caprylyl Glycol | | | | | | |
| | Chlorphenesin | | | | | | |
| ARG-DPG | Dipotassium | 1.00% | 0.50% | 0.06% | 0.04% | 0.07% | 0.10% |
| | Glycyrrhizate | | | | | | |
| AA2G | Ascorbyl Glucoside | 1.55% | 1.50% | 3.00% | 2.00% | 1.76% | 1.98% |
| Drago-Calm 674463 | Water (Aqua); | 0.24% | 0.50% | 0.11% | 0.20% | 0.13% | 0.40% |
| | Glycerin; Avena sativa | | | | | | |
| | (Oat) Kernel Extract | | | | | | |
| Cytobiol Lumin-Eye | Butylene Glycol; | 2.50% | 6.50% | 3.85% | 1.50% | 4.00% | 4.20% |
| | Water (Aqua); | | | | | | |
| | Niacinamide; Fraxinus | | | | | | |
| | excelsior Bark Extract; | | | | | | |
| | Silanetriol; Potassium | | | | | | |
| | Citrate | | | | | | |
| Haloxyl | Water (Aqua); | 1.00% | 2.50% | 2.00% | 1.50% | 2.75% | 1.75% |
| | Glycerin; Steareth-20; | | | | | | |
| | N- | | | | | | |
| | Hydroxysuccinimide; | | | | | | |
| | Chrysin; Palmitoyl | | | | | | |
| | Oligopeptide; | | | | | | |
| | Palmitoyl | | | | | | |
| | Tetrapeptide-7 | | | | | | |
| Matrixyl 3000 | Glycerin; Water | 1.25% | 2.00% | 3.20% | 2.50% | 3.00% | 1.75% |
| | (Aqua); Butylene | | | | | | |
| | Glycol; Carbomer | | | | | | |
| | Polysorbate 20; | | | | | | |
| | Palmitoyl | | | | | | |
| | Oligopeptide; | | | | | | |
| | Palmitoyl | | | | | | |
| | Tetrapeptide-7 | | | | | | |
| Fragrance - “Silky | Fragrance (Parfum) | 0.01% | 0.05% | 0.07% | 0.03% | 0.06% | 0.01% |
| Skin” - #6110985 | | | | | | | |
| ChronoSphere | Silica; Polyurethane- | 1.25% | 3.50% | 1.98% | 2.00% | 3.00% | 0.50% |
| Opticals Brite | 40; Green 5 | | | | | | |

TABLE 5
Representative FnBu Compositions.

| Trade Name | INCI Name | Range | 12 | 13 | 14 | 15 | 16 | 17 | 18 | 19 | 20 |
|---|---|---|---|---|---|---|---|---|---|---|---|
| perfluoro(n- | perfluoro(n- | 1-10% | 2.00% | 3.00% | 5.00% | 4.00% | 4.50% | 6.00% | 45.50% | 47.75% | 50.00% |
| butylcyclohexane) | butylcyclohexane) | | | | | | | | | | |
| Deionized Water | Water (Aqua) | 50-75% | 66.90% | 72.15% | 61.55% | 62.55% | 67.61% | 59.55% | 41.93% | 38.45% | 37.50% |
| Carbopol Ultrez-10 | Carbomer | 0-5% | 0.50% | 1.00% | 0.35% | 0.00% | 0.00% | 0.00% | 0.20% | 0.70% | 0.35% |
| Zemea | Propanediol | 0.5-5% | 2.00% | 1.00% | 1.50% | 2.00% | 3.00% | 3.50% | | | |
| Dissolvine Na-2-P | Disodium EDTA | 0-1% | 0.20% | 0.30% | 0.10% | 0.00% | 0.00% | 0.00% | 0.02% | 0.05% | 1.00% |
| Avocadin (HU-25) | Persea gratissima | 0-2% | 0.90% | 0.50% | 0.75% | 0.00% | 0.00% | 0.00% | 0.00% | 0.00% | 0.00% |
| | (Avocado) Oil | | | | | | | | | | |
| | Unsaponifiables | | | | | | | | | | |
| Shea Butter Refined | Butyrospermum parkii | 1-5% | 1.50% | 2.00% | 2.55% | 1.20% | 1.50% | 2.00% | 0.00% | 0.00% | 0.00% |
| or “Ultra Refined” | (Shea Butter) | | | | | | | | | | |
| Simulsol 165 | Glyceryl Stearate | 0-10% | 3.50% | 3.00% | 5.00% | 0.00% | 0.00% | 0.00% | 0.00% | 0.00% | 0.00% |
| | PEG-100 Stearate | | | | | | | | | | |
| Myritol 312 | Caprylic/Capric | 0-5% | 0.00% | 0.00% | 0.00% | 3.00% | 3.75% | 3.30% | 0.00% | 0.00% | 0.00% |
| | Triglyceride | | | | | | | | | | |
| Botanol MO | Limnanthes alba | 0-5% | 0.00% | 0.00% | 0.00% | 2.00% | 0.40% | 1.00% | 0.00% | 0.00% | 0.00% |
| | (Meadowfoam) | | | | | | | | | | |
| | Seed Oil | | | | | | | | | | |
| Sodium Hydroxide, | Sodium Hydroxide | 0-1% | 0.00% | 0.00% | 0.00% | 0.25% | 0.10% | 0.60% | 0.00% | 0.00% | 0.00% |
| Pellets, NF | | | | | | | | | | | |
| Mikrokill Cos | Phenoxyethanol | 0.1-5% | 2.00% | 1.00% | 0.45% | 3.00% | 0.70% | 1.00% | 0.00% | 0.00% | 0.00% |
| | Caprylyl Glycol | | | | | | | | | | |
| | Chlorphenesin | | | | | | | | | | |
| ARG-DPG | Dipotassium | 0.05-1% | 0.70% | 0.10% | 0.45% | 0.10% | 0.20% | 0.70% | 0.00% | 0.00% | 0.00% |
| | Glycyrrhizate | | | | | | | | | | |
| AA2G | Ascorbyl Glucoside | 0-5% | 0.00% | 0.00% | 0.00% | 2.00% | 1.00% | 1.50% | 0.00% | 0.00% | 0.00% |
| Matrixyl 3000 | Glycerin | 0-5% | 3.00% | 3.10% | 1.00% | 0.00% | 0.00% | 0.00% | 0.00% | 0.00% | 0.00% |
| | Water (Aqua) | | | | | | | | | | |
| | Butylene Glycol | | | | | | | | | | |
| | Carbomer | | | | | | | | | | |
| | Polysorbate 20 | | | | | | | | | | |
| | Palmitoyl Oligopeptide | | | | | | | | | | |
| | Palmitoyl | | | | | | | | | | |
| | Tetrapeptide-7 | | | | | | | | | | |
| AMP Ultra PC 2000 | Aminomethyl | 0-2% | 0.20% | 0.10% | 0.50% | 0.00% | 0.00% | 0.00% | 0.65% | 0.45% | 0.35% |
| | Propanol | | | | | | | | | | |
| Olivem 1000 | Sorbitan Olivate | 0.01-5% | 1.00% | 0.85% | 0.50% | 0.75% | 1.00% | 1.50% | 0.00% | 0.00% | 0.00% |
| | Cetearyl Olivate | | | | | | | | | | |
| Lanette 16 | Cetyl Alcohol | 0-5% | 0.50% | 1.00% | 2.30% | 0.00% | 0.00% | 0.00% | 0.00% | 0.00% | 0.00% |
| Lipohate GC | Caprylic/Capric | 0-5% | 2.00% | 2.75% | 3.15% | 0.00% | 0.00% | 0.00% | 0.00% | 0.00% | 0.00% |
| | Triglyceride | | | | | | | | | | |
| Jojoba Oil Colorless | Simmondsia chinensis | 0-5% | 1.50% | 1.50% | 2.50% | 0.00% | 0.00% | 0.00% | 0.00% | 0.00% | 0.00% |
| | (Jojoba) Seed Oil | | | | | | | | | | |
| Avocado Oil | Persea gratissima | 0-5% | 2.00% | 1.50% | 3.00% | 0.00% | 0.00% | 0.00% | 0.00% | 0.00% | 0.00% |
| | (Avocado) Oil | | | | | | | | | | |
| Dimethisil DM-350 | Dimethicone | 0-5% | 4.00% | 1.00% | 2.75% | 0.00% | 0.00% | 0.00% | 0.00% | 0.00% | 0.00% |
| Brookosome ACE | Water (Aqua) | 0-1% | 0.10% | 0.30% | 0.20% | 0.00% | 0.00% | 0.00% | 0.00% | 0.00% | 0.00% |
| | Phospholipids | | | | | | | | | | |
| | Tocopheryl Acetate | | | | | | | | | | |
| | Retinyl Palmitate | | | | | | | | | | |
| | Ascorbyl Palmitate | | | | | | | | | | |
| Kalpariane | Caprylic/Capric | 0.5% | 2.50% | 1.00% | 3.00% | 0.00% | 0.00% | 0.00% | 0.00% | 0.00% | 0.00% |
| | Triglyceride | | | | | | | | | | |
| | Alaria esculenta | | | | | | | | | | |
| | extract | | | | | | | | | | |
| Dow Corning 9041 | Dimethicone | 0-5% | 3.00% | 2.85% | 3.40% | 0.00% | 0.00% | 0.00% | 0.00% | 0.00% | 0.00% |
| Silicone Elastomer | Dimethicone | | | | | | | | | | |
| Blend | Crosspolymer | | | | | | | | | | |
| Avicel PC-591 | Microcrystaline | 0-5% | 0.00% | 0.00% | 0.00% | 2.20% | 1.50% | 2.40% | 0.00% | 0.00% | 0.00% |
| | Cellulose | | | | | | | | | | |
| | Cellulose Gum | | | | | | | | | | |
| Glycerine 99.7% | Glycerine | 0-5% | 0.00% | 0.00% | 0.00% | 3.75% | 2.75% | 3.00% | 0.00% | 0.00% | 0.00% |
| USP | Water (Aqua) | | | | | | | | | | |
| Keltrol CG | Xanthan Gum | 0-5% | 0.00% | 0.00% | 0.00% | 1.00% | 0.20% | 0.90% | 0.00% | 0.00% | 0.00% |
| Dermofeel PA-3 | Water (Aqua) | 0-2% | 0.00% | 0.00% | 0.00% | 0.10% | 0.50% | 0.75% | 0.00% | 0.00% | 0.00% |
| | sodium phytate | | | | | | | | | | |
| LexFeel Natural | Heptyl undecylenate | 0-5% | 0.00% | 0.00% | 0.00% | 2.00% | 2.40% | 1.60% | 0.00% | 0.00% | 0.00% |
| Oilwax | Olea europaea | 0-5% | 0.00% | 0.00% | 0.00% | 1.40% | 1.00% | 1.80% | 0.00% | 0.00% | 0.00% |
| | (Olive) Fruit Oil | | | | | | | | | | |
| | Hydrogenated | | | | | | | | | | |
| | Olive Oil | | | | | | | | | | |
| | Olea europaea | | | | | | | | | | |
| | (Olive) Oil | | | | | | | | | | |
| | Unsaponifiables | | | | | | | | | | |
| Lanette O | Cetearyl Alcohol | 0-5% | 0.00% | 0.00% | 0.00% | 1.50% | 1.20% | 1.00% | 0.00% | 0.00% | 0.00% |
| Arophira | Caprylic/Capric | 0-2% | 0.00% | 0.00% | 0.00% | 0.50% | 0.10% | 0.80% | 0.00% | 0.00% | 0.00% |
| | triglyceride | | | | | | | | | | |
| | Hydrogenated | | | | | | | | | | |
| | Vegetable Oil | | | | | | | | | | |
| | Crithmum maritimum | | | | | | | | | | |
| | extract | | | | | | | | | | |
| BV-OSC | Tetrahexyldecyl | 0-2% | 0.00% | 0.00% | 0.00% | 0.90% | 1.00% | 1.50% | 0.00% | 0.00% | 0.00% |
| | ascorbate | | | | | | | | | | |
| Montanov 68 | Cetearyl Alcohol | 0-5% | 0.00% | 0.00% | 0.00% | 3.00% | 3.80% | 3.20% | 0.00% | 0.00% | 0.00% |
| | Cetearyl Glucoside | | | | | | | | | | |
| Sodium Citrate USP | Sodium Citrate | 0-2% | 0.00% | 0.00% | 0.00% | 0.80% | 0.79% | 0.30% | 0.00% | 0.00% | 0.00% |
| B-White | Water (Aqua) | 0-5% | 0.00% | 0.00% | 0.00% | 2.00% | 1.00% | 2.10% | 0.00% | 0.00% | 0.00% |
| | Butylene Glycol | | | | | | | | | | |
| | Hydrogenated Lecithin | | | | | | | | | | |
| | Sodium Oleate | | | | | | | | | | |
| | Disodium EDTA | | | | | | | | | | |
| | Oligopeptide-68 | | | | | | | | | | |
| | Glycerin | | | | | | | | | | |
| | Glycine Soja | | | | | | | | | | |
| | (Soybean) Oil | | | | | | | | | | |
| Hydrolite-5 #516751 | Pentylene glycol | 0-5.% | 0.00% | 0.00% | 0.00% | 0.00% | 0.00% | 0.00% | 0.70% | 3.00% | 1.00% |
| Ajidew NL-50 | Sodium PCA | 0-5% | 0.00% | 0.00% | 0.00% | 0.00% | 0.00% | 0.00% | 0.80% | 0.50% | 1.00% |
| | Water (Aqua) | | | | | | | | | | |
| Schercemol 318 Ester | isopropyl isostearate | 0-10% | 0.00% | 0.00% | 0.00% | 0.00% | 0.00% | 0.00% | 3.00% | 2.00% | 2.70% |
| Botanistat PF-64 | Phenoxyethanol | 0.5% | 0.00% | 0.00% | 0.00% | 0.00% | 0.00% | 0.00% | 0.80% | 1.00% | 0.85% |
| | Caprylyl glycol | | | | | | | | | | |
| | Ethylhexylglycerin | | | | | | | | | | |
| | Hexylene glycol | | | | | | | | | | |
| Derm SRC-PF | Water (Aqua) | 0-2% | 0.00% | 0.00% | 0.00% | 0.00% | 0.00% | 0.00% | 1.70% | 0.10% | 0.50% |
| | Glucosemine HCl | | | | | | | | | | |
| | Pisum sativum | | | | | | | | | | |
| | (Pea) extract | | | | | | | | | | |
| | Bambusa vulgaris | | | | | | | | | | |
| | leaf/stern extract | | | | | | | | | | |
| Volasil 995 | Cyclopentasiloxane | 0-10.00% | 0.00% | 0.00% | 0.00% | 0.00% | 0.00% | 0.00% | 3.20% | 4.00% | 3.00% |
| Amphisol A | Aminomethyl | 1-10.00% | 0.00% | 0.00% | 0.00% | 0.00% | 0.00% | 0.00% | 1.50% | 2.00% | 1.75% |
| | Propanol | | | | | | | | | | |

TABLE 6
Representative FnBu Composition Characteristics

| | Composition 1 | Composition 2 | Composition 3 | Composition 4 | Composition 5 |
|---|---|---|---|---|---|
| Color: | Light blue to | Grayish Green | White to off- | White to off- | White to off- |
| | light green | | white | white | white |
| Appearance | Opaque, | Opaque, semi- | Opaque, | Opaque, semi- | Opaque, |
| | viscous cream | viscous lotion | viscous cream | viscous lotion | viscous lotion |
| pH at 25° C. | 6.00-7.00 | 8.00-8.80 | 5.00-5.80 | 5.80-8.50 | 5.50-6.50 |
| Viscosity at | 10,000- | 18,000- | 20,000- | 13,000- | 15,000- |
| 25° C. | 20,000 cps | 30,000 cps | 30,000 cps | 18,000 cps | 25,000 cps |
| | (RVT: Spindle | (Brookfield RT | (initial); | (Brookfield | (Brookfield |
| | 5 @ 10 rpm) | DV-II+; | 35,000- | RT DV-II+; #8 | RT DV-II+: |
| | | Spindle #8 | 75,000 cps | @10 rpm, | Spindle #6 |
| | | @10 rpm) | (final) | 1 min) | @ 10 rpm, |
| | | | (RVT: Spindle | | 1 min) |
| | | | T-D @ 10 rpm) | | |
| Specific | 1.14-1.18 | 1.02-1.22 | 0.97-1.01 | 1.01-1.04 | 1.26-1.34 |
| gravity at | | | | | |
| 25° C. | | | | | |
| Total | Less than | Less than | Less than | Less than | Less than |
| Aerobic | 100 cfu/g | 100 cfu/g | 100 cfu/g | 100 cfu/g | 100 cfu/g |
| Plate | | | | | |
| Count: | | | | | |
| Yeast & | Less than | Less than | Less than | Less than | Less than |
| Mold: | 100 cft/g | 100 cft/g | 100 cft/g | 100 cft/g | 100 cft/g |
| P. Aeruginosa: | Absent | — | — | — | — |
| S. Aureus: | Absent | — | — | — | — |
| Percent | 19.80%-22.00% | 20.85-22.85% | 3.50-24.50% | 19.88-21.99% | 5.50-7.50% |
| Solids at | | (2 g heat to | (2 g heat to | (2 g heat to | (weigh approx |
| 130° C. | | 130° C.) | 130° C.) | 130° C.) | 2 grams, heat |
| | | | | | to 130° C.) |

### Example 2

### Manufacturing the Perfluorocarbon Cream

A perfluorocarbon cream (composition 1 of Table 6) according to the present invention can be manufactured in 5 phases according to Table 7 below:

TABLE 7

| Item No. | Trade Name | Processing Container |
|---|---|---|
| Phase A | | |
| 1 | Deionized (DI) Water | Main Processing Tank |
| 2 | Carbopol Ultrez-10 | Main Processing Tank |
| 3 | Zemea | Main Processing Tank |
| 4 | Dissolvine Na-2-P | Main Processing Tank |
| Phase B | | |
| 5 | Avocadin | Auxiliary Tank |
| 6 | Shea Butter “Ultra | Auxiliary Tank |
| | Refined” | |
| 7 | Stearic Acid | Auxiliary Tank |
| 8 | Amphisol A | Auxiliary Tank |
| 9 | Simulsol 165 | Auxiliary Tank |
| 10 | Dow Corning 200 | Auxiliary Tank |
| | Fluid 350 CST | |
| 11 | Volasil 995 | Auxiliary Tank |
| 12 | Myritol 312 | Auxiliary Tank |
| 13 | KP-545 | Auxiliary Tank |
| 14 | Soline | Auxiliary Tank |
| 15 | Botanol MO | Auxiliary Tank |
| Phase C | | |
| 16 | Deionized Water | Auxiliary Tank |
| 17 | Sodium Hydroxide, | Auxiliary Tank |
| | Pellets, NF | |
| Phase D | | |
| 18 | Mikrokill Cos | Main Processing Tank |
| 19 | ARG-DPG | Main Processing Tank |
| 20 | AA2G | Main Processing Tank |
| 21 | Drago-Calm 674463 | Main Processing Tank |
| 22 | Cytobiol Lumin-Eye | Main Processing Tank |
| 23 | Haloxyl | Main Processing Tank |
| 24 | Matrixyl 3000 | Main Processing Tank |
| 25 | perfluoro (n- | Main Processing Tank |
| | butylcyclohexane) | |
| 26 | Fragrance - “silky | Main Processing Tank |
| | Skin” - #6110985 | |
| Phase E | | |
| 27 | Deionized Water | Auxiliary Tank |
| 28 | ChronoSphere | Auxiliary Tank |
| | Opticals Brite | |

The manufacturing procedures:
1. Phase A:
   a. Add Item No. 1 (Deionized water) into the main processing tank.
   b. Start high speed mixing.
   c. Add Item No. 2.
   d. Mix until completely dispersed.
   e. Heat to 80° C.-85° C.
   f. Add Item Nos. 3 and 4.
   g. Mix until uniform.
   h. Maintain temperature.
2. Phase B:
   a. In a separate tank, add Item Nos. 5-15.
   b. Heat to 80° C.-85° C.
   c. Mix until all the solids are completely dissolved.
   d. Add Phase B to Phase A.
   e. Mix until uniform.
3. Phase C:
   a. In a separate container, add Item Nos. 16 and 17.
   b. Mix until all the solids are completely dissolved.
   c. Add Phase C to the main tank.
   d. Mix for 30 minutes until uniform.
   e. Cool to 40° C.
4. Phase D:
   a. At 40° C., add Item Nos. 18-26, mixing well after each addition.
5. Phase E:
   a. In a separate container, add Item Nos. 27 and 28.
   b. Mix until completely homogeneous.
   c. Add homogenous mixture to the main tank.
   d. Mix until uniform. Homogenize if necessary.
6. Continue mixing and cooling to 35° C. Mix for 20 minutes or until uniform.

A perfluorocarbon lotion (composition 2 of Table 6) according to the present invention can be manufactured in according to the steps below:
1. Add DI Water into the main processing tank and begin moderate mixing. Add Carbopol Ultrez-10, heat to 80° C., and mix until uniform.
2. Add Zemea and Dissolvine Na-2-P in the given order and mix until uniform.
3. In a separate vessel, add Avocadin HU-25, Shea Butter Ultra Refined, Stearic Acid, Amphisol A, Simulsol 165, Dimethisil DM-350, Volasil 995, Myritol 312, KP-545, Soline and Botanol MO, heat to 80° C. then mix until uniform. At 80° C., add contents of the separate vessel to the main processing tank and mix until uniform
4. In another separate vessel, add Sodium Hydroxide Pellets NF and DI water then mix until uniform. At 80° C., add the contents of this separate vessel to the main processing tank and mix until uniform. Cool to 40° C.
5. At 40° C., add Mikrokill Cos and ARG-DPG in the given order to the main processing tank, mix until uniform
6. In another separate vessel, add DI water, Sodium Hydroxide Pellets NF and AA2G, mix until uniform. At 40° C., add the contents of this vessel into the main processing tank. Mix until uniform.
7. At 40° C., add Drago-Calm 674463, Cytobiol Lumin-Eye, Haloxyl, Matrixyl 3000, Fragrance “Silky Skin” and perfluoro(n-butylcyclohexane) in the given order to the main processing tank. Mix until uniform.
8. In another separate vessel, add DI water and ChronoSphere Opticals Brite. Mix until uniform. At 40° C., add the contents of this separate vessel to the main processing tank and mix until uniform. Continue mixing and for 20 minutes or until uniform.

A perfluorocarbon cream (composition 3 of Table 6) according to the present invention can be manufactured in according to the steps below:
1. Add DI water into the main processing tank and begin high speed mixing.
2. Add Carbopol Ultrez-10 into the processing tank, disperse until uniform, heat to 80° C., and mix until uniform.
3. At 80° C., add Dissolvine Na-2-P and Zemeato the main processing tank. Mix until uniform. Add AMP Ultra PC 2000 to the main processing tank, mix until uniform.
4. In a separate vessel, add Simulsol 165, Olivem 1000, Lanette 16, Shea Butter Ultra Refined, Avocadin HU-25, Lipohate GC, Jojoba Oil Colorless, Avocado Oil and Dimethisil DM-350, heat to 80° C. and mix until uniform. At 80° C., add the contents of the separate vessel to the main processing tank. Mix the contents of the main processing tank for 20 to 30 minutes until completely uniform. Cool to 40° C. Adjusting speed if necessary.
5. In another separate vessel, add DI water and ARG-DPG, mix until uniform. At 40° C., add the contents of this separate vessel to the main processing tank. Mix until uniform.
6. At 40° C., add Mikrokill Cos, Brookosome ACE, Kalpariane, Matrixyl 3000, Dow Corning 9041 Silicone Elastomer Blend and perfluoro(n-butylcyclohexane) in the given order to the main processing tank. Mix until uniform.
7. Continue mixing and for 20 minutes or until uniform.

A perfluorocarbon lotion (composition 4 of Table 6) according to the present invention can be manufactured in according to the steps below:
1. Add DI water into the main processing tank water and begin moderate speed mixing. Add Avicel PC-591, mix until uniform.
2. In a separate vessel, add Zemea, Glycerine 99.7% USP and Keltrol CG. Mix until uniform. Add the contents of the separate vessel to the main processing tank, mix until uniform. Heat to 75° C.
3. At 75° C., add Dermofeel PA-3 and ARG-DPG into the main processing tank, mix until uniform.
4. In a separate vessel, add LexFeel Natural, Myritol 312, Oilwax, Botanol MO, Shea Butter Refined, Olivem 1000, Lanette O, Arophira, BV-OSC and Montanov 68, heat to 75° C. and mix until uniform. At 75° C., add the contents of this separate vessel to the main processing tank, mix until uniform.
5. At 75° C., add Mikrokill Cos to the main processing tank, mix until uniform. Homogenize for 10 minutes, cool to 40° C.
6. In another separate vessel, add DI water, AA2G, Sodium Citrate USP and Sodium Hydroxide Pellets NF, heat to 40° C., mix until uniform. At 40° C., add the contents of this separate vessel to the main processing tank, mix until uniform.
7. At 40° C., add B-White and perfluoro(n-butylcyclohexane) in the given order into the main processing tank, mix until uniform.
8. Continue mixing and for 20 minutes or until uniform.

A perfluorocarbon lotion (composition 5 of Table 6) according to the present invention can be manufactured in according to the steps below:
1. In a main processing tank equipped with a propeller mixer and side sweep, add DI water. Begin high speed mixing.
2. Sprinkle Carbopol Ultrez 20 into the main processing tank and mix the contents until completely uniform and free of lumps. Heat to 85° C.
3. Add Dissolvine Nz-2-P, Hydrolite-5 #616751 and Ajidew NL-50 into the main processing tank and mix until uniform.
4. In a separate vessel, add Amphisol A and Schercemol 318 Ester, heat to 85° C. and then mix until uniform. Add the contents of this separate vessel into the main processing tank and mix until uniform. Cool to 70° C.
5. Slowly add Volasil 995 and perfluoro(n-butylcyclohexane) in the given order. Mix well after each addition, mix until uniform.
6. Premix DI water and AMP Ultra PC 2000, and add to the main processing tank. Mix the contents until homogenous. Cool to 40V, adjusting mixing speed if necessary.
7. Add Botanistat PF-64 and Derm SRC—PF into the processing tank and mix until uniform.
8. Continue cooling to 35° C. and mix for 20 minutes or until uniform.

### Example 3

### Manufacturing Stable Perfluorocarbon Gels

### Example 3A

Stable gels composed of water, a surfactant (Pluronic F-68 or Pluronic F-127), and perfluoro(n-butylcyclohexane) (FnBu) are produced in accordance with procedures described below.

### Materials

1. Pluronic F-68: [Sigma-Aldrich P1300-500G Batch #097K0116 CAS 9003-11-6];
2. Pluronic F-127: [Sigma-Aldrich P2443-250G Batch #038K0113 CAS 9003-11-6];
3. n-butylperfluorocyclohexane (Perfluoro(n-butylcyclohexane), or FnBu)
4. Ethyl Alcohol, absolute, 200 proof, 99.5%, A.C.S. reagent: [ACROS 61509-0040, CAS 64-17-5];
5. Distilled H₂O;
6. 20-100 mL glass beakers;
7. 5-20 mL glass beakers;
8. 20-50 mL Corning centrifuge tubes;
9. 5-60 mL Teflon capped, glass jars;
10. OMNI Macro ES Homogenizer;
11. 750 Watt, 20 kHz Ultrasonic Processor;
12. Fisherbrand Spoonulet Lab Spoon;
13. Spatula;
14. Pipet;
15. 5 mL NORM-JECT® luer lock, airtight syringe; and
16. B-D® 26 gauge ½ inch, luer lock, Precision Glide® syringe needle.

### Experimental Procedures

### Gel 1

16.25 g of distilled water is weighed into a 100 mL glass beaker. 20 g of FnBu is added to the beaker followed by 5 g of F-127. The contents of the beaker are then manually stirred with a spatula for 30 seconds. The tip of an OMNI Macro ES Homogenizer is submerged into the contents of the beaker, and the stirred mixture is homogenized for approximately 5 minutes at 4000 rpm. The homogenized mixture is poured into a 50 mL Corning centrifuge tube. The procedure is then repeated three times in order to prepare 4 centrifuge tubes. All 4 centrifuge tubes are centrifuged in an IEC Clinical Centrifuge for 30 minutes. The off-fluid of each tube is poured out and weighed separately. The gel remaining in each tube is scooped out using a Fisherbrand Spoonulet Lab Spoon and weighed into a 60 mL Teflon capped, glass jar.

### Gel 2

16.25 g of distilled water is weighed into a 100 mL glass beaker. 20 g of FnBu is added to the beaker followed by 5 g of F-127. The contents of the beaker are then manually stirred with a spatula for 30 seconds. The tip of a 750 Watt, 20 kHz Ultrasonic Processor is submerged into the contents of the beaker, and the stirred mixture is sonicated for approximately 5 minutes at 20% amplitude. The sonicated mixture is poured into a 50 mL Corning centrifuge tube. The procedure is then repeated three times in order to prepare 4 centrifuge tubes. All 4 centrifuge tubes are centrifuged in an IEC Clinical Centrifuge for 30 minutes. The off-fluid of each tube is poured out and weighed separately. The gel remaining in each tube is scooped out using a Fisherbrand Spoonulet Lab Spoon and weighed into a 60 mL Teflon capped, glass jar.

### Gel 3

16.25 g of distilled water is weighed into a 100 mL glass beaker. 20 g of FnBu is added to the beaker followed by 5 g of F-68. The contents of the beaker are then manually stirred with a spatula for 30 seconds. The tip of an OMNI Macro ES Homogenizer is submerged into the contents of the beaker, and the stirred mixture is homogenized for approximately 5 minutes at 4000 rpm. The homogenized mixture is poured into a 50 mL Corning centrifuge tube. The procedure is then repeated three times in order to prepare 4 centrifuge tubes. All 4 centrifuge tubes are centrifuged in an IEC Clinical Centrifuge for 30 minutes. The off-fluid of each tube is poured out and weighed separately. The gel remaining in each tube is scooped out using a Fisherbrand Spoonulet Lab Spoon and weighed into a 60 mL Teflon capped, glass jar.

### Determination of Perfluorocarbon Yields

Approximately 5 g of each gel is placed individually into 20 mL glass beakers. Using a pipet, 7.00 g, 6.32 g, and 5.48 g of ethanol are added to each beaker containing Gels 1-3, respectively. Each gel/ethanol mixture is stirred for 5 minutes using a spatula. Each stirred mixture is allowed to sit for 3 minutes in order for two layers, an aqueous layer and a perfluorocarbon layer, to separate. The perfluorocarbon layer is removed from the beaker using a 5 mL syringe with a 26 gauge, 2 inch syringe needle. The weight of the perfluorocarbon layer is recorded. This weight divided by the initial (˜5 g) gel weight for each gel sample gives the perfluorocarbon yield for each gel.

### Results

The perfluorocarbon yield is defined as the percentage of perfluorocarbon added during the preparation that remained as part of the recovered gel. The percent gel yield is defined as the total weight of recovered gel relative to the total weight of components added during preparation. The perfluorocarbon yields for gels 1-3 is between 30-95% and gel yields are between 40-50%.

### Example 3B

Table 8 shows four embodiments of the subject invention (Gels 1-4).

TABLE 8

| | grams/gram of gel | | | |
|---|---|---|---|---|
| | Gel 1 | Gel 2 | Gel 3 | Gel 4 |
| Component | 75, 25 - T | 75, 25 - H | (PQ)² - T | (PQ)² - H |
| perfluoro(n- | 85.980% | 86.726% | 85.980% | 86.726% |
| butylcyclohexane) | | | | |
| Distilled Water | 10.277% | 10.366% | 10.277% | 10.366% |
| Pluronic ® F-68 | 0.307% | 0.310% | 0.307% | 0.310% |
| Pluronic ® L-35 | 2.446% | 2.467% | 2.446% | 2.467% |
| Polyquaternium-6 | 0.000% | 0.000% | 0.248% | 0.033% |
| Polyquaternium-7 | 0.743% | 0.099% | 0.495% | 0.066% |
| EDTA | 0.248% | 0.033% | 0.248% | 0.033% |

Pluronic® is a trade name of BASF Corporation (Mt. Olive, N.J.). Pluronic F-68 and Pluronic L-35 are hydroxyl-terminated ethylene oxide-propylene oxide block copolymers. They have the general formula: HO(C₂H₄O)ₐ(C₃H₆O)_{b}(C₂H₄O)_{c}H. Subscripts a and c are usually about equal and subscript b is usually 15 or higher. F-68 is a solid with a molecular weight of about 8400; L-35 is a liquid with a molecular weight of about 1900.

The chemical structures for Polyquaternium-6 and Polyquaternium-7 are shown below:

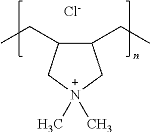

Polyquaternium 6 ionic surfactant/preservative
Poly(diallyldimethylammonium chloride)

### (CAS No. 26062-79-3) (Nalco Merquat® 100)

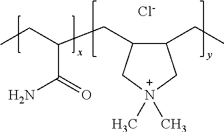

Polyquaternium 7 ionic surfactant/preservative
Poly(acrylamide-co-diallyldimethylammonium chloride)

### (CAS No. 26590-05-06) (Nalco Merquat® 740)

These materials are sold by several companies including Nalco Company of Naperville, Ill. Both chemicals contain highly polar dimethylammonium chloride quaternary salts. There are many other polyquat salts as shown in Table 9. However, not all are used as preservatives.

TABLE 9

| Product | CAS RN |
|---|---|
| polyquaternium 1 | 75345-27-6 |
| polyquaternium 2 | 68555-36-2 |
| polyquaternium 4 | 92183-41-0 |
| polyquaternium 5 | 26006-22-4 |
| polyquaternium 6 | 26062-79-3 |
| polyquaternium 7 | 26590-05-6 |
| polyquaternium 10 | 68610-92-4 |
| polyquaternium 11 | 53633-54-8 |
| polyquaternium 12 | 68877-50-9 |
| polyquaternium 13 | 68877-47-4 |
| polyquaternium 14 | 27103-90-8 |
| polyquaternium 15 | 35429-19-7 |
| polyquaternium 16 | 95144-24-4 |
| polyquaternium 22 | 53694-17-0 |
| polyquaternium 24 | 107987-23-5 |
| polyquaternium 28 | 131954-48-8 |
| polyquaternium 31 | 136505-02-7 |
| polyquaternium 32 | 35429-19-7 |
| polyquaternium 33 | 69418-26-4 |
| polyquaternium 37 | 26161-33-1 |
| polyquaternium 44 | 150599-70-5 |
| polyquaternium 46 | 174761-16-1 |
| polyquaternium 57 | 9004-97-1 |

EDTA is ethylene diamine tetraacetic acid. The disodium salt and tetrasodium salt of EDTA are more frequently used than the tetraacid as cosmetic preservatives. However, these salts (in fact, any ionizable salt) will break the gel or prevent the gel from forming.

The concentrations of the three preservatives are based either on the total basic gel weight (including the FnBu), designated “-T” gels or the concentration is based on the weight of the water and Pluronics only, designated “-H” gels. The 75, 25-T gel (Gel 1) contains 7500 ppm of Polyquat-7 and 2500 ppm of EDTA, both based on the total formulation weight including the FnBu. Gel (PQ)²-H (Gel 4) contains 2500 ppm PQ-6, 5000 ppm PQ-7, and 2500 ppm EDTA—each based on the weight of the aqueous phase in the gel only.

### Gel Formation and Processing

The formation of gels 1-4 proceeds by first mixing the aqueous phase components (distilled water, F-68, L-35, and the preservatives of choice) in a glass, polyethylene, PET, or 316 stainless steel vessel. The mixture is homogenized for about 5 minutes with a rotor/stator homogenizer at 10,000-35,000 RPM. The homogenizer can be handheld for small samples (<2 L), a bench top unit for larger (2-5 L) samples, or a larger, floor mounted version of these mixers for commercial scale production (>5 L).

During mixing of the aqueous phase, not all components need be completely soluble. The F-68 has limited solubility in water and homogenization mostly disperses this solid as very fine particles once the saturation limit for F-68 in water has been reached. Similarly, high concentrations of EDTA can result in a fine particle dispersion after the solubility limit for EDTA in water has been attained (˜500 ppm in water at 20° C.)

After homogenization of the aqueous phase mixture, the perfluorocarbon (PFC) is added either in aliquots or slowly and continuously over the course of the next 10-30 minutes of high speed homogenization. Gel formation tends to occur only at the latter stages of PFC addition. The gels that form do not require centrifugation and separation as taught by Moore in U.S. Pat. No. 4,569,784, which is hereby incorporated by reference herein.

Continued homogenization past the 25-30 minutes typical for gel formation creates more viscous gels. For some formulations, the long term stability of the gel improves with longer mixing. The formulations which will exhibit this behavior can be determined by trial and error. Other PFC gels can be obtained by this process.

### Factors Affecting Gel Formation and Processing

There are many compounds and materials that are incompatible with the disclosed gels.

### Alcohols

Trace levels of alcohols will immediately or eventually cause the gel to break. This behavior is observed with trace amounts of methanol, ethanol, isopropanol, tecopherol, chlorhexidine digluconate, chlorphenesin, and glycerol. It appears that any compound having a primary, secondary, or tertiary hydroxyl or phenolic group will break the gel or prevent the formation of the gel.

### Highly Ionized Salts

Highly ionized compounds (salts) can prevent the formation of the gel or break the gel once formed. While low levels (<5000 ppm) of EDTA can be incorporated successfully, the di- and tetrasodium salts of EDTA prevent formation. Tap water contains sufficient levels of ions to break the gel in a period of 1-24 hours after contact. While polymeric quaternary ammonium compounds have been successfully added, benzalkonium chloride will prevent gel formation at ppt levels or lower. If highly ionized salts contact the gel after formation, the salts can break the gel even if not mechanically mixed into the bulk. It is often sufficient for gel destruction to contact one surface of the gel with a quiescent aqueous puddle of the offensive compound. Once the gel begins to break, it tends to continue to unravel over a period of hours to days.

### Highly Nonpolar Solid Surfaces

Highly nonpolar solid surfaces are incompatible with these gels and will break the gels quickly or over time. This occurs whenever the perfluorocarbon can “wet” a solid surface and form a film of the pure PFC. The film tends to segregate gravitationally and sink slowly to the bottom of the vessel holding the gel. This process “renews” or frees the surface to contact more gel and separate more PFC. The process continues slowly until a large part of the gel has broken and formed two distinct phases. This behavior is observed for packaging films having heat seal lacquer coatings and for Teflon® surfaces. Teflon is an especially aggressive gel breaker. Thus far, it appears that glass, polyethylene, PET, nylon, and other non-PFC wettable surfaces are compatible with the gels.

### Metal Surfaces

Certain metal surfaces are incompatible with gels but for differing reasons. Aluminum surfaces are easily wetted by the PFC and cause separation and eventually breaking of the gels. 304 stainless steel, unlike 316 stainless, is attacked and corroded by the gels. The surface of 304 stainless is passivated by an oxide coating that is easily breached by the chloride anion of the polyquat salts. Once breached, the surface is attacked by the EDTA and corroded. It is anticipated that other incompatible metals will be observed with more testing. Clearly, the choice of materials of construction is important for commercial production of these gels.

### Packaging Materials

Some packaging materials are inappropriate for the gels. In particular, those plastics that are highly permeable to water will be poor choices since loss of the aqueous phase by diffusion through the plastics will degrade and eventually break the gels. A good example is PET. A single layer of PET will allow water in the gel to escape. However, if PET is sandwiched with polyethylene or polypropylene, the poor solubility of water in the polyolefins will lower the permeation loss rate to an acceptable level and the gel will remain secure.

### Example 4

### Oxygen-Rich Perfluorocarbon Compositions Improve Skin Appearance

The periodic topical application of an oxygen-rich composition comprising perfluoro(n-butylcyclohexane) formulated according to table 10 to subjects' skin improves the skin's overall appearance.

TABLE 10

| Component | Wt % |
|---|---|
| perfluoro(n-butylcyclohexane) | 86.00 |
| Water | 10.25 |
| Pluronic ® L35 (Poloxamer 105) | 2.45 |
| Gluconolactone, Sodium Benzoate, | 1.00 |
| Calcium Gluconate | |
| Pluronic ® F-108 (Poloxamer 388) | 0.30 |

### Fitzpatrick Wrinkle Assessment Scale (FWAS)

The investigator assesses the degree of facial wrinkling and elastosis at all visits. The investigator performs a live facial assessment of the subject using the FWAS, a 10-point categorical scale corresponding to 0 (None, no wrinkling or elastosis), 1-3 (Mild, fine wrinkles and fine textual changes with subtly accentuated skin lines), 4-6 (Moderate, fine to moderate depth wrinkles, moderate number of lines, and distinct popular elastosis), and 7-10 (Severe, fine to deep wrinkles, numerous lines with or without redundant skin folds, and multipapular and confluent elastosis). In order to be enrolled in the clinical study, subjects are required to have a FWAS grade of Mild to Moderate corresponding to a FWAS score of 1-6.

A significant portion of the study subjects show at least one grade improvement on the Fitzpatrick Wrinkle Assessment Scale (FWAS) beginning at the four-week time point. There is a significant difference between 4, 5, 6, 7 and 8-week FWAS scores and those from baseline. By the conclusion of the study of subjects with mild-to-moderate facial wrinkles, most of subjects exhibit at least one-grade improvement on the FWAS with many showing at least a two grade improvement.

Global Aesthetic Improvement Scale (GALS)

The subject and the investigator assess data for both impressions of how the treatment had an effect on the overall appearance of facial skin using the CAIS, a 5-point categorical scale consisting of the responses worse, no change, improved, much improved, or very much improved. The subject and investigator complete a CAIS at visits 2-9 by comparing a photograph from the current visit to a photograph from the subject's baseline visit.

There is a significant increase in favorable responses for the subject Global Aesthetic Improvement Scale (CAIS) score beginning with the five-week time point and continuing through the eight-week time point, suggesting that the subjects perceive improvement of their skin's overall appearance, or at least the maintenance of their skin's overall appearance. Investigator CAIS scores also show significant improvement beginning at the three-week time point and continuing through the study. By the conclusion of the study, majority of the subjects experience at least one grade of improvement compared to baseline according to the investigator's CAIS. In addition, most of the subjects have at least one grade of improvement on their self-perceived CAIS score.

### Skin Replica Data

Skin Replica silicon profilometry is performed at baseline, 4-weeks (Visit 5) and 8-weeks (Visit 9). The major and minor lines are measured by 8 parameters separated into two groups of 4. Group A parameters define the luminance along a set of 10 equal length parallel lines (or passes) running across the replica and are parallel to the direction of lighting. The variations within the luminance are treated as indications of the skin's roughness, representing major lines and are analyzed using surface roughness statistics. Group B parameters represent minor lines assess the replica image area by dividing it into 10 equal width bands (or sub areas). The shadow-like features are detected in each of the bands according to their luminance values as compared to those less than the detection threshold.

The Skin Replica data suggests that the PFC composition had a mild smoothing effect on the subjects' fine lines and wrinkles.

### Discussion

As the study progresses, more subjects exhibit improvement in fine lines and skin elastosis. Also, more subjects report improvement of their appearance with the length (time) they are applying the PFC composition. The number of subjects responding ‘improved’, ‘much improved’ or ‘very much improved’ continue to increase through the different time points increasing in significance at each subsequent visit.

Investigator CAIS data also suggest more subjects demonstrating improvement in overall appearance the longer they are applying the PFC composition.

FWAS, subject CAIS and investigator CAIS results suggest the majority of subjects show signs of improvement at the four-week time point.

Skin Replica data results show smoothing effect of minor fine lines around the Crow's Feet area of the eye for those subjects applying the PFC composition over the 8-week duration of the study. These results are significant for the breadth and shadows parameters suggesting a mild smoothing effect. The treatment has effects on the collagen matrix of the skin. The PFC composition acts at the cellular level, providing subtle softening effects for the fine lines around the eye.

The application of the PFC composition (twice daily) improves the appearance of fine lines and overall texture of the skin after a period of 4-6 weeks.

### Example 5

### Perfluoro(n-butylcyclohexane) Composition Is Effective Relieving Pruritus Secondary to a Histamine Prick Test

### Materials and Method:

The standard histamine prick test is utilized to create a localized area of acute allergic response (classic wheal and flare reaction). The method involves placing a small drop of histamine on each subject's forearm and wrist. To initiate the allergic reaction, a sharp needle is used to “prick” the skin just under the histamine. The subjects each have two standardized histamine prick tests. A perfluoro(n-butylcyclohexane) composition is then applied in blinded fashion at the one site 10 minutes after the reaction is initiated and after 20 minutes for the other. Extent of pruritus is assessed by asking each subject to judge the severity of the itching at the site. In addition, the Investigator scores the edema and erythema at each site.

### Histamine Prick Test (Darsow, 2000):

1. Causes an immediate wheal (edema) and flare (erythema) reaction that is graded 0-4.
2. Subject is then treated with either a moisturizing cream or perfluoro(n-butylcyclohexane).

Primary End Points: Subject perception of itching at each prick test sites.

Secondary End Point: Decrease in size of edema and erythema secondary to prick test.

Exploratory End Point: Onset of anti-pruritic effect compared to potent steroid cream (Elocon)

### Results:

1. Most subjects develop pruritus secondary to the histamine test.
2. Most of the subjects who developed pruritus report less itching with the application of the PFC composition than at the untreated reaction at 10 and 20 minutes.
3. Wheal and flare (edema and erythema) appear smaller after the application of the PFC composition.
4. PFC composition works worked faster than a potent steroid cream.

### Discussion:

This study demonstrates the efficacy of the perfluoro(n-butylcyclohexane) composition in decreasing histamine-induced pruritus. The PFC composition decreases the size of the histamine induced edema and erythema. It also has a faster onset of action than some topical steroids in reducing itch.

### Example 6

### Perfluoro(n-butylcyclohexane) Composition For Cosmetic Applications

### Example 6A

A perfluoro(n-butylcyclohexane) composition as described herein is topically administered to the periocular skin a subject in need thereof. Topical administration of the perfluoro(n-butylcyclohexane) composition is effective to improve the overall appearance of the subject's periocular skin by reducing the appearance of or the severity of fine lines, wrinkles, puffiness, dark (under-eye) circles, bags and/or dark blemishes in the subjects' skin.

### Example 6B

A perfluoro(n-butylcyclohexane) composition as described herein is topically administered to the periocular skin of a subject. Topical administration of the perfluoro(n-butylcyclohexane) composition is effective to increases oxygen delivery to the periocular skin of the subject.

### Example 6C

A perfluoro(n-butylcyclohexane) composition as described herein is administered topically to a subject. Specifically, the perfluoro(n-butylcyclohexane) composition is administered topically to the skin on the subject. The perfluoro(n-butylcyclohexane) composition increases oxygen level and oxygen tension in the skin tissue. In addition, the perfluoro(n-butylcyclohexane) composition reduces the appearance of skin imperfection associated with aging including fine lines and wrinkles. Also, the perfluoro(n-butylcyclohexane) composition improves the firmness of the skin where applied.

### Example 6D

A perfluoro(n-butylcyclohexane) composition as described herein mixed with caffeine is administered topically to a subject. Specifically, the perfluoro(n-butylcyclohexane) composition is administered topically to the cellulite-affected skin on the subject. The perfluoro(n-butylcyclohexane) composition increases oxygen level and oxygen tension in the skin tissue. In addition, the perfluoro(n-butylcyclohexane) composition reduces the appearance the cellulite where applied.

### Example 7

### Treatment of Acne and Rosacea

### Example 7A

A perfluoro(n-butylcyclohexane) composition as described herein is topically administered to the skin of a subject suffering from acne at the site of the acne. Topical administration of the perfluoro(n-butylcyclohexane) composition is effective to treat the subject's acne. Acne reduction is noticeable, as is a reduction in skin appearance characteristics associated with acne.

### Example 7B

A perfluoro(n-butylcyclohexane) composition as described herein is topically administered to the skin a subject suffering from acne vulgaris at the site of the acne vulgaris. Topical administration of the perfluoro(n-butylcyclohexane) composition is effective to reduce acne-scarring in the subject by reducing the severity of existing acne vulgaris and preventing or reducing the severity of further acne vulgaris in the subject.

### Example 7C

A perfluoro(n-butylcyclohexane) composition as described herein is topically administered a subject suffering from a Propionibacterium acnes infection of a skin follicle of the subject. The composition is applied to the skin follicle or the area of skin surrounding the skin follicle. Topical administration of the perfluoro(n-butylcyclohexane) composition is effective to reduce the Propionibacterium acnes infection of the skin follicle of the subject.

### Example 7D

A perfluoro(n-butylcyclohexane) composition as described herein is topically administered to the skin of a subject suffering from a Propionibacterium acnes infection of the dermis of the subject. The composition is applied to the skin comprising the infected dermis. Topical administration of the perfluoro(n-butylcyclohexane) composition is effective to reduce the Propionibacterium acnes proliferation in the dermis of the subject.

### Example 7E

A perfluoro(n-butylcyclohexane) composition as described herein is topically administered to the skin of a subject susceptible to acne. Topical administration of the perfluoro(n-butylcyclohexane) composition is effective to prevent or reduce the subject's acne.

### Example 7F

A perfluoro(n-butylcyclohexane) composition as described herein is topically administered to the skin of a subject wherein there are Propionibacterium acnes in and/or on the skin. Topical administration of the perfluoro(n-butylcyclohexane) composition is effective to kill Propionibacterium acnes in and/or on the skin of the subject.

In the above examples the administration of the composition is one, two or three times per day. The administration can be repeated daily for a period of one, two, three or four weeks, or longer. The administration can be continued for a period of months or years as necessary.

### Example 7G

A perfluoro(n-butylcyclohexane) composition as described herein is topically administered to the skin of a subject suffering from rosacea at the site of the rosacea. Topical administration of the composition comprising the perfluorocarbon or oxygenated perfluorocarbon is effective to treat the subject's rosacea. Rosacea reduction is noticeable, as is a reduction in skin appearance characteristics associated with rosacea.

### Example 8

### Wound and Burn Healing and Scar Prevention and Reduction

### Example 8A

A perfluoro(n-butylcyclohexane) composition as described herein is administered topically to a subject. Specifically, the perfluoro(n-butylcyclohexane) composition is administered topically to a wound on the subject. The perfluoro(n-butylcyclohexane) composition increases oxygen level and oxygen tension in the wound tissue. In addition, the perfluoro(n-butylcyclohexane) composition accelerates wound healing.

### Example 8B

A perfluoro(n-butylcyclohexane) composition as described herein is administered topically to a subject. Specifically, the perfluoro(n-butylcyclohexane) composition is administered topically to a burn wound on the subject. The perfluoro(n-butylcyclohexane) composition increases oxygen level and oxygen tension in the burnt tissue and surrounding tissue. In addition, the perfluoro(n-butylcyclohexane) composition accelerates the healing of the burn wound.

### Example 8C

A perfluoro(n-butylcyclohexane) composition as described herein is administered topically to a subject. Specifically, the perfluoro(n-butylcyclohexane) composition is administered topically to a wound or a scar on the subject. The perfluoro(n-butylcyclohexane) composition increases oxygen level and oxygen tension in the wound or scarred tissue. In addition, the perfluoro(n-butylcyclohexane) composition accelerates wound healing and ameliorates and reduces the appearance of the scar.

### Example 9

### Pruritus and Psoriasis

### Example 9A

### Allergic Pruritus

A perfluoro(n-butylcyclohexane) composition as described herein is administered topically to a subject suffering fromitching secondary to insect bites. Relief of itching secondary to insect bites occurs almost immediately and lasts approximately 3 hours.

A perfluoro(n-butylcyclohexane) composition as described herein is administered topically to a subject suffering from dermatitis. Contact dermatitis is resolved rapidly with the administration a perfluoro(n-butylcyclohexane) composition as described herein.

Use of a perfluoro(n-butylcyclohexane) composition as described herein for treating atopic dermatitis results in improvement in itching lasting 3-4 hours. Erythematous lesions also improves.

### Example 9B

### Psoriasis

A perfluoro(n-butylcyclohexane) composition as described herein is administered to a subject afflicted with psoriasis.

The administration the PFC composition is effective to alleviate a symptom of psoriasis, relieve pruritus associated with the psoriasis, improve the appearance of the skin where the composition is applied, reduce subject's perceived itching and increase anti-inflammatory activity on the skin where the composition is applied.

The administration of the PFC composition is also effective to reduce edema, erythema and erythematous lesions.

### Example 9C

### Pruritus

A perfluoro(n-butylcyclohexane) composition as described herein is administered to a subject afflicted with pruritus.

The administration the PFC composition is effective to treat the pruritus, reduce subject's perceived itching and increase anti-inflammatory activity on the skin where the composition is applied.

The administration of the PFC composition is also effective to reduce edema, erythema and erythematous lesions.

### Example 9D

### Pruritus Resulting from Xerosis

A perfluoro(n-butylcyclohexane) composition as described herein is administered to a subject afflicted with xerosis.

The administration the PFC composition is effective to treat the pruritus resulting from xerosis, reduce subject's perceived itching and increase anti-inflammatory activity on the skin where the composition is applied.

The administration of the PFC composition is also effective to reduce edema, erythema and erythematous lesions.

### Example 9E

### Pruritus Resulting From Atopic Dermatitis (AD)

A perfluoro(n-butylcyclohexane) composition as described herein is administered to a subject afflicted with atopic dermatitis.

The administration the PFC composition is effective to treat the pruritus resulting from the dermatitis, decrease release of histamine, decrease acute exacerbations, reduce subject's perceived itching and increase anti-inflammatory activity on the skin where the composition is applied.

The administration of the PFC composition is also effective to reduce edema, erythema and erythematous lesions.

### Example 9F

### Pruritus Resulting from Contact Dermatitis

A perfluoro(n-butylcyclohexane) composition as described herein is administered to a subject afflicted with contact dermatitis.

The administration the PFC composition is effective to treat the pruritus resulting from the dermatitis, decrease release of histamine, decrease acute exacerbations, reduce subject's perceived itching and increase anti-inflammatory activity on the skin where the composition is applied.

The administration of the PFC composition is also effective to reduce edema, erythema and erythematous lesions.

### Example 9G

### Pruritus Induced by Histamine

A perfluoro(n-butylcyclohexane) composition as described herein is administered to a subject afflicted with histamine-induced pruritus.

The administration the PFC composition is effective to treat the histamine-induced pruritus, decrease release of histamine, decrease acute exacerbations, reduce subject's perceived itching and increase anti-inflammatory activity on the skin where the composition is applied.

The administration of the PFC composition is also effective to reduce edema, erythema and erythematous lesions.

### Example 9H

### Dermatological Allergic Response

A perfluoro(n-butylcyclohexane) composition as described herein is administered to a subject afflicted with a dermatological allergic response.

The administration the PFC composition is effective to alleviate a symptom of the dermatological allergic response, treat pruritus resulting from the dermatological allergic response, reduce subject's perceived itching and increase anti-inflammatory activity on the skin where the composition is applied. The administration of the PFC composition is also effective to reduce edema or erythema associated with the allergic response.

### Example 9I

### Inflammatory Skin Condition

A perfluoro(n-butylcyclohexane) composition as described herein is administered to a subject afflicted with an inflammatory skin condition.

The administration the PFC composition is effective to alleviate a symptom of the inflammatory skin condition, treat pruritus resulting from the inflammatory skin condition, reduce subject's perceived itching and increase anti-inflammatory activity on the skin where the composition is applied. The administration of the PFC composition is also effective to reduce edema or erythema associated with the inflammatory skin condition.

### Example 9J

### Edema

A perfluoro(n-butylcyclohexane) composition as described herein is administered to the skin of a subject afflicted with edema.

The administration the PFC composition is effective reduce the edema.

### Example 9K

### Erythema

A perfluoro(n-butylcyclohexane) composition as described herein is administered to the skin of a subject afflicted with erythema.

The administration the PFC composition is effective reduce the erythema.

### Example 10

### Sexual Enhancement

### Example 10A

A perfluoro(n-butylcyclohexane) composition as described herein is administered topically to sex organs of a human male subject. Local oxygen tension and nocturnal erections are evaluated. Changes in Quality of life (QOL) data is also collected and assessed.

Oxygen level and oxygen tension in the tissue increases. In addition, Quality of life of the subject improves. Moreover, the perfluorocarbon is well tolerated and has no toxicity.

### Example 10B

A perfluoro(n-butylcyclohexane) composition as described herein is topically administered to sex organs of male and female human subjects. The perfluoro(n-butylcyclohexane) composition is administered once or twice daily. Local oxygen tension and nocturnal erections (in males) are evaluated. Changes in Quality of life (QOL) data is also collected and assessed.

Oxygen level and oxygen tension in the tissue is increases. In addition, Quality of life of the subject improves.

### REFERENCES

1. “Dermatitis” The Merck Manual, 17th ed. Mark H. Beers, MD, Robert Berkow, MD, eds. Whitehouse Station, N.J.: Merck Research Labs, 1999, pp. 786-789.
2. “Pruritus” The Merck Manual, 17th ed. Mark H. Beers, MD, Robert Berkow, MD, eds. Whitehouse Station, N.J.: Merck Research Labs, 1999, pp. 782.
3. “Psoriasis” The Merck Manual, 17th ed. Mark H. Beers, MD, Robert Berkow, MD, eds. Whitehouse Station, N.J.: Merck Research Labs, 1999, pp. 816-818.
4. Bekyarova, G., et al. (1997) “Suppressive effects of FC-perluorocarbon emulsion on enhanced oxidative haemolysis in the early postburn phase.” Burns. (23)2: 117-121.
5. Butler G. (2009) “Therapeutic Effect of Hyperbaric Oxygen in Psoriasis Vulgaris: Two Case Reports and Review of the Literature.” J. Med Case Rep. 3:7023.
6. Darsow, (2000) “Processing of Histamine-Induced Itch in the Human Cerebral Cortex: A Correlation Analysis with Dermal Reactions.” Journal of Investigative Dermatology 115:1029-1033.
7. Davis S. (007) “Topical Oxygen Emulsion, A Novel Wound Therapy.” Arch Dermatology. 143:1252-1256.
8. Davis, Stephen C., et al. (2007) “Topical Oxygen Emulsion: A Novel Wound Therapy” Arch Dermatol. 143(10): 1252-1256.
9. Eady et al., (1989) “Erythromycin resistant propionibacteria in antibiotic treated acne patients: Association with therapeutic failure” Br J. Dermatol. 1989 July; 121(1):51-7.
10. Kaneda, Megan M., et al. (2009) “Perfluorocarbon nanoemulsions for quantitative molecular imaging and targeted therapeutics” Ann Biomed Eng. 37(10) October 2009. NDN 230-1024-9131-6.
11. Lehmler H. (2008) “Anti-inflammatory Effects of Perfluorocarbon Compounds.” Exp Rev Res Med. 2:273-289.
12. McDongh P. (2001) “Perflubron Emulsion Reduces Inflammation during Extracorporeal Circulation.” J. Surg. Res. 99:7-16.
13. Sarafidis H. (2008) “Perfluorochemical Augmented Delivery Attenuates Inflammation in the Immature Lung.” J. NeoNat Perna Med. 2008:1.
14. Shen, Yao, et al. (2007) “Carnosine attenuates mast cell degranulation and histamine release induced by oxygen-glucose deprivation” Cell Biochemistry and Function. 26(3):334-338.
15. Shen, Yao, et al. (2007) “Carnosine attenuates mast cell degranulation and histamine release induced by oxygen-glucose deprivation” Cell Biochemistry and Function. 26(3):334-338.
16. Thiboutot et al., (1997) “Acne. An overview of clinical research findings” Dermatol Clin. 1997 January; 15(1):97-109.
17. U.S. Pat. No. 4,490,351, issued Dec. 25, 1984 to Leland Clark Jr.
18. U.S. Pat. No. 4,569,784, issued Feb. 11, 1986 to Robert E. Moore.
19. U.S. Pat. No. 4,857,304, issued Aug. 15, 1989 to Ishiwatari, et al.
20. U.S. Pat. No. 5,643,601, issued Jul. 1, 1997 to Gross, et al.
21. U.S. Patent Application Publication No. U.S. 2010-0144861, published Jun. 10, 2010 (Huvard et al.).

1. A perfluorocarbon composition comprising 1-90 wt % perfluoro(n-butylcyclohexane) relative to the total weight of the composition.

2-9. (canceled)

10. The perfluorocarbon composition of claim 1, comprising 1) perfluoro(n-butylcyclohexane), 2) dipotassium glycyrrhizate and 3) a mixture comprising phenoxyethanol, caprylyl glycol and chlorphenesin.

11. The perfluorocarbon composition of claim 10, further comprising a second mixture comprising sorbitan olivate and cetearyl olivate.

12. The perfluorocarbon composition of claim 11, comprising 1-10 wt % perfluoro(n-butylcyclohexane), 0.05-1 wt % dipotassium glycyrrhizate, 0.1-5 wt % of the mixture comprising phenoxyethanol, caprylyl glycol and chlorphenesin, 0.01-5 wt % of the second mixture comprising sorbitan olivate and cetearyl olivate and 15-50 wt % water.

13-14. (canceled)

15. The perfluorocarbon composition of claim 1, comprising perfluoro(n-butylcyclohexane), cetyl phosphate and cyclopentasiloxane.

16. The perfluorocarbon composition of claim 15, further comprising isopropyl isostearate.

17. The perfluorocarbon composition of claim 16, comprising 35-60 wt % perfluoro(n-butylcyclohexane), 0.5-5 wt % cetyl phosphate, 1-5 wt % cyclopentasiloxane, 1-5 wt % isopropyl isostearate and 35-60 wt % water.

18. (canceled)

19. The perfluorocarbon composition of claim 1, comprising 1) perfluoro(n-butylcyclohexane), 2) ascorbyl glucoside, 3) a first mixture comprising butylene glycol, water, niacinamide, Fraxinus excelsior bark extract, silanetriol and potassium citrate, 4) a second mixture comprising water, glycerin, steareth-20, N-hydroxysuccinimide, chrysin, palmitoyl oligopeptide and palmitoyl tetrapeptide-7 and 5) a third mixture comprising glycerin, water, butylene glycol, carbomer, polysorbate 20, palmitoyl oligopeptide and palmitoyl tetrapeptide-7.

20. (canceled)

21. The perfluorocarbon composition of claim 1, comprising 1-55 wt % perfluoro(n-butylcyclohexane) relative to the total weight of the composition.

22-24. (canceled)

25. The perfluorocarbon composition of claim 19, wherein the ascorbyl glucoside is 1-10 wt % relative to the total weight of the composition.

26. The perfluorocarbon composition of claim 19, wherein the first mixture is 1-10 wt % relative to the total weight of the composition.

27. The perfluorocarbon composition of claim 19, wherein the second mixture is 1-10 wt % relative to the total weight of the composition.

28. The perfluorocarbon composition of claim 19, wherein the third mixture is 1-10 wt % relative to the total weight of the composition.

29. The perfluorocarbon composition of claim 19, comprising 1) 15-50 wt % perfluoro(n-butylcyclohexane), 2) 0.1-5 wt % ascorbyl glucoside, 3) 1-10 wt % of the first mixture comprising butylene glycol, water, niacinamide, Fraxinus excelsior bark extract, silanetriol and potassium citrate, 4) 0.1-5 wt % of the second mixture comprising water, glycerin, steareth-20, N-hydroxysuccinimide, chrysin, palmitoyl oligopeptide and palmitoyl tetrapeptide-7, 5) 0.1-5 wt % of the third mixture comprising glycerin, water, butylene glycol, carbomer, polysorbate 20, palmitoyl oligopeptide and palmitoyl tetrapeptide-7 and 6) 15-50 wt % water.

30. (canceled)

31. The perfluorocarbon composition of claim 1, characterized by it having a viscosity of 5,000-80,000 cps at 25° C.

32-34. (canceled)

35. The perfluorocarbon composition of claim 1, characterized by it having a specific gravity of 0.9-1.82.

36. (canceled)

37. The perfluorocarbon composition of claim 1, further comprising a pharmaceutically acceptable carrier or a cosmetic carrier.

38. The perfluorocarbon composition of claim 1, in the form of a liquid, cream, lotion or gel.

39. A method of delivering oxygen to the skin of a subject comprising topically administering to the skin the perfluorocarbon composition of claim 1 effective to deliver oxygen to the skin.

40. A method of increasing the firmness of the skin or reducing the appearance of fine lines, wrinkles or scars in a subject, or improving the appearance of the skin of a subject, or treating a wound, a burn injury, pruritus, psoriasis, acne or rosacea in a subject suffering therefrom, comprising topically administering to the skin of the subject the perfluorocarbon composition of claim 1 effective to increase the firmness of the subject's skin or reduce the appearance of fine lines, wrinkles or scars on the subject's skin, or effective to improve the appearance of the skin, or effective to treat the subject's wound, burn injury, pruritus, psoriasis, acne or rosacea.

41-57. (canceled)
